(19) United States
(12) Patent Application Publication
(10) Pub. No.: US 2011/0224515 A1
(43) Pub. Date: Sep. 15, 2011

(54) REPLACEABLE MICRONEEDLE CARTRIDGE FOR BIOMEDICAL MONITORING

(75) Inventors: Jose Mir, Rochester, NY (US); John Spoonhower, Webster, NY (US); John A. Agostinelli, Rochester, NY (US); Larry Demejo, Rochester, NY (US); Kamal K. Sarbadhikari, Geneseo, NY (US)

(73) Assignee: SensiVida Medical Technologies, Inc., Henrietta, NY (US)

(21) Appl. No.: 12/877,755

(22) Filed: Sep. 8, 2010

Related U.S. Application Data
(60) Provisional application No. 61/276,116, filed on Sep. 8, 2009.

(51) Int. Cl. A61B 5/157; A61B 5/15

(52) U.S. Cl. 600/317; 600/575; 600/309; 600/365

(57) ABSTRACT

A replaceable microneedle array for a biomedical monitor is disclosed. The microneedle array includes a plurality of moveable microneedles coated with at least one chemical sensing material coupled with a porous material. The microneedle array also includes a substrate defining wells to house the microneedles. The microneedle array further includes at least one restoring spring element coupled between each microneedle and the substrate such that each of the plurality of microneedles is held at least partially in an associated well.

### RELATED APPLICATION

This application claims priority to U.S. Provisional Patent Application No. 61/276,116 filed Sep. 8, 2009 and entitled, “COMPACT MINIMALLY INVASIVE BIOMEDICAL MONITOR USING IMAGE PROCESSING”. U.S. Provisional Patent Application No. 61/276,116 is also hereby incorporated by reference in its entirety.

### FIELD

This technology generally relates to replaceable test cartridges for biomedical monitors, and more specifically to replaceable test cartridges having an array of minimally invasive microneedles.

### BACKGROUND

Existing methods for measuring blood glucose and other blood and/or interstitial fluid-based parameters suffer from a number of disadvantages. For example, the well-known fingerstick monitor requires the use of a fine lancet which invasively pierces the skin to draw blood for subsequent analysis. Unfortunately, as a result of the discomfort and inconvenience of the process, compliance tends to be low, especially for younger and older patients. Repeated lancet piercing can also lead to sensitivity and/or hardening of the subject's skin since fingertips are one of the body's most sensitive regions. Furthermore, fingerstick-based monitors only provide a sampled measurement of the subject's blood chemistry even though glucose levels fluctuate rapidly after meals. This creates problems especially for diabetics who need to monitor their glucose levels over 5 times a day, exacerbating usage issues for the patient. With growing numbers of patients requiring regular blood/fluid based biomedical testing, patients and physicians have been searching for a more continuous monitoring process that is less painful or even painless, less invasive, more convenient, automatable, and which requires little or no periodic calibration.

As described in The Pursuit of Non-Invasive Glucose: “Hunting the Deceitful Turkey” by John L. Smith, a large number of attempts to bring a non-invasive glucose monitor to market have been made, and so far none has been successful. Generally, the many methods that have been pursued exhibit poor accuracy because of glucose interferents and other uncontrolled variables.

Microneedle technology provides a useful minimally-invasive method to sample body fluids. Due to their small size, microneedles can pierce skin and sample minute quantities of blood or interstitial fluid with minimal impact and/or pain to the subject. In spite of their advantages for reducing patient discomfort, many microneedle systems described in the prior art are still somewhat invasive since they extract and transport blood or interstitial fluid from the patient for the measurement. Furthermore, the small quantity of fluid sampled by microneedles can lead to great variability in concentration measurements.

Implanted in vivo sensors have also been developed to sample blood chemistry. Such implanted sensors have the advantage of not requiring blood extraction. Unfortunately, however, long term use of implanted sensors is hampered by a process known as “bio-fouling”. Bio-fouling refers to changes in device characteristics caused by its interaction with the in vivo environment as a result of the device's long term presence in the subject. At best, bio-fouling requires frequent calibration to compensate for these changes; more often than not these changes are irreversible and require device replacement. Implanted in vivo sensors also require an accommodation period, typically hours, after implantation before useful monitoring can begin. In addition, implanted sensors are inserted subcutaneously into a very complex environment comprising a large number of anatomical structures including hair follicles, sebaceous tissue, sweat glands, nerve fibers, and more. The implanted sensors are blind to their precise local environments. Accuracy achieved using continuous glucose monitoring with implanted sensors is not adequate for therapeutic use.

### SUMMARY

A replaceable microneedle array for a biomedical monitor is disclosed. The microneedle array includes a plurality of moveable microneedles coated with at least one chemical sensing material coupled with a porous material. The microneedle array also includes a substrate defining wells to house the microneedles. The microneedle array further includes at least one restoring spring element coupled between each microneedle and the substrate such that each of the plurality of microneedles is held at least partially in an associated well.

This technology provides a number of advantages. A biomedical monitor may be configured to receive the convenient replaceable microneedle array. Such biomedical monitors may be removably attached to a subject and are able to make multiple sequential blood chemistry measurements. The biomedical monitor provides a highly useful device configuration and convenient fabrication process for dense arrays of individually actuated microneedles having integral chemical sensors. The compact wearable device can sample body chemistry without extracting a significant amount of blood or interstitial fluid either during or after the microneedle is inserted in the subject. Consequently, the degree of invasiveness and risk of contamination is reduced, while improving the hygiene of the process. Due to their high multiplicity, microneedles with integral chemical sensing material may be inserted in the subject in sequence over an extended period of time, each chemical sensing element being required to make measurements for only a short time period. The use of each microneedle for a limited time will eliminate the effect of bio-fouling. Sequential actuation of a multiple microneedles provides the ability for long term monitoring. Control of the serial actuation process can be programmed for a specific monitoring schedule, making the process practically continuous, if desired, and convenient for a subject. Due to their dense spacing and integrated actuation capability, many measurements may be made for extended time periods using a compact device worn by the subject as a small patch or chip. The biomedical monitor may be configured to sense chemicals which are naturally produced and/or found in a subject's body as well as chemicals which a subject has been exposed to, for example harmful toxins or biological components.

### BRIEF DESCRIPTION OF THE DRAWINGS

FIGS. 1A-1D schematically illustrate one embodiment of a biomedical monitor.

FIGS. 2A-2K schematically illustrate embodiments of sensing materials coated on microneedles, in cross-sectional views, for use with a biomedical monitor.

FIGS. 3A-3F schematically illustrate embodiments of multiple sensing regions coated on microneedles, in cross-sectional views, for use with a biomedical monitor.

FIG. 4 schematically illustrates an enlarged view of the highlighted region from FIG. 3C showing one embodiment of a scattered light path when the microneedle is used with a biomedical monitor.

FIGS. 5A-5C schematically illustrate embodiments of multiple sensing regions coated on microneedles, in top views, for use with a biomedical monitor.

FIGS. 6A and 6B illustrate embodiments of a microneedle array for use with a biomedical monitor in a needle-up view.

FIG. 7A schematically illustrates a cross-sectional view of a portion of the microneedle array from FIG. 6A.

FIG. 7B schematically illustrates a cross-sectional view of another embodiment of a microneedle array.

FIGS. 8A and 8B schematically illustrate a cross-sectional view of a portion of further embodiments of a microneedle array having a calibration position.

FIGS. 9A-9B show images captured by an imaging sensor showing a coated microneedle before and after insertion into a test environment.

FIG. 9C shows a difference image of the microneedle image captured by the imaging sensor after insertion into the test environment (FIG. 9B) subtracting the microneedle image captured by the imaging sensor before insertion into the test environment (FIG. 9A).

FIG. 9D shows the sampled color change region from the image of FIG. 9C with background subtraction.

FIG. 9E shows an example of an extracted color change region.

FIGS. 10A-10C separately illustrate pixel histograms of the sampled color change region for red, green, and blue channels.

FIG. 11 is a plot of the effect of sampled sensor area on the coefficient of variation of measured intensity for pixels of each color within the color change region.

FIG. 12 illustrates the expected statistical error as a function of sampled area when color ratios red to blue, and red to green, before and after insertion, are used to characterize the response.

FIG. 13 shows expected statistical distributions of data points on a Clark Error Grid for several sampled area sizes.

FIG. 14 illustrates one embodiment of a method for monitoring at least one biomedical characteristic.

FIG. 15 schematically illustrates a cross-sectional view of another embodiment of a microneedle array.

It will be appreciated that for purposes of clarity and where deemed appropriate, reference numerals have been repeated in the figures to indicate corresponding features. Illustrations are not necessarily drawn to scale. While spatial imaging methods and a replaceable microneedle cartridge for biomedical monitoring are described herein by way of example for several embodiments and illustrative drawings, those skilled in the art will recognize that the system and method are not limited to the embodiments or drawings described. It should be understood, that the drawings and detailed description thereto are not intended to limit embodiments to the particular form disclosed. Rather, the intention is to cover all modifications, equivalents and alternatives falling within the spirit and scope of the appended claims. Any headings used herein are for organizational purposes only and are not meant to limit the scope of the description or the claims. As used herein, the word “may” is used in a permissive sense (i.e., meaning having the potential to), rather than the mandatory sense (i.e., meaning must). Similarly, the words “include”, “including”, and “includes” mean including, but not limited to.

### DETAILED DESCRIPTION

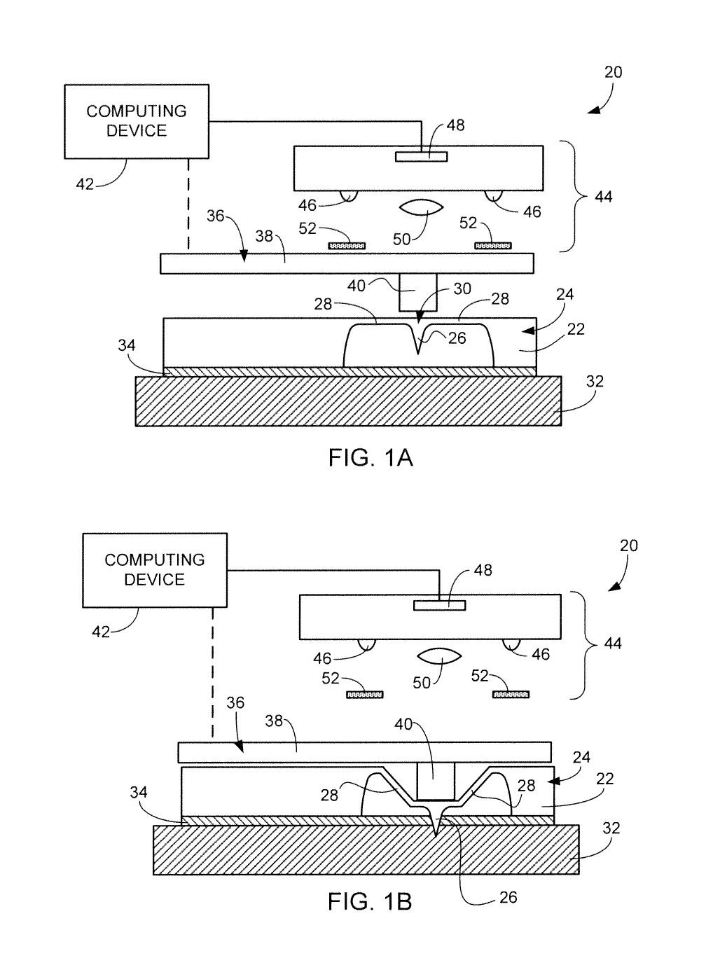

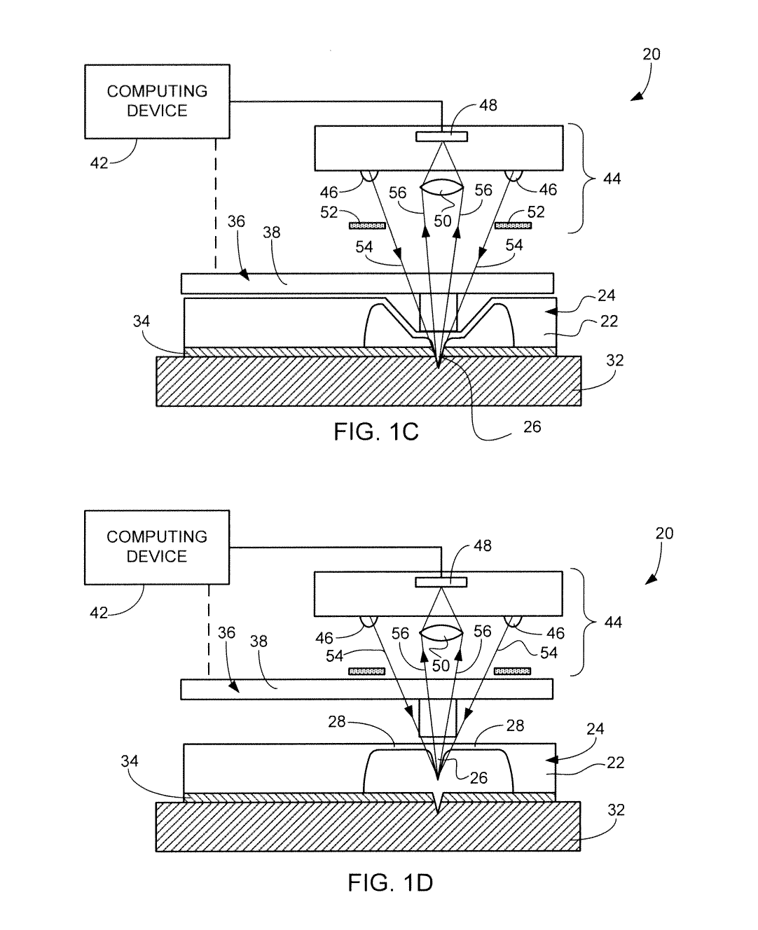

FIG. 1A schematically illustrates one embodiment of a biomedical monitor 20. The biomedical monitor 20 has a microneedle array 22. The microneedle array 22 may include a substrate 24 which has been micro-machined or precision molded to define one or more microneedles 26 supported by at least one restoring spring element 28. The one or more microneedles 26 should be dimensioned to penetrate the subject's stratum corneum and reach the underlying interstitial fluid or capillary network. The microneedles 26 can be very fine, on the order of 5-50 microns in diameter at the tip, and from 20-2000 microns in height, although smaller or larger diameter and/or height needles may be used in other embodiments. The at least one restoring spring element 28 could be patterned directly out of the substrate 24 material or out of a layer having desirable mechanical properties that has been deposited onto substrate 24. Alternatively, restoring spring 28 may also be patterned out of one or more materials in a multi-material substrate where additional materials have been deposited on or bonded to the substrate 24. For example, an oxidized substrate may be etched to form the one or more microneedles 26 out of silicon and a restoring spring 28 out of either the silicon dioxide layer or a combination of the silicon dioxide layer and the silicon layer. Similarly, using technology such as SOI (silicon-on-insulator), a silicon dioxide microneedle may be etched and the restoring spring be patterned out of the silicon layer. Although not illustrated in this embodiment, other embodiments may include positional sensors on the restoring springs 28 for use in determining the deflection of the microneedle 26. The at least one restoring spring 28 can be patterned in a number of geometries such as a spiral spring, a cantilever structure, or other geometries as long as they provide the freedom of movement that allows microneedle 26 to protrude far enough out of a plane defined by substrate 24 in order to penetrate a subject's skin to a desired depth.

A number of substrate 24 and/or microneedle 26 materials maybe used, e.g. silicon, silicon dioxide, silicon nitride, all commonly used in microfabrication or, in general, dielectrics, plastics, metals, glass, quartz, or sapphire. The microneedle 26 and a base 30 of the microneedle 26 are preferably transparent, but may be translucent in some embodiments. Another option would be to have the bulk material of the microneedle be transparent, while its surface be scattering or translucent. Several fabrication techniques for the one or more microneedles 26 are disclosed in the literature, such as photolithography, reactive ion etching, isotropic etching (e.g. for glass), plastic molding, water jet milling, and others may be used. The one or more microneedles 26 may be solid or hollow. The microneedle 26 cross-sections may be variable or constant, and can take on a variety of cross-sectional shapes, including, but not limited to square, circular, triangular, and grooved. Other embodiments of microneedles 26 may even be corrugated.

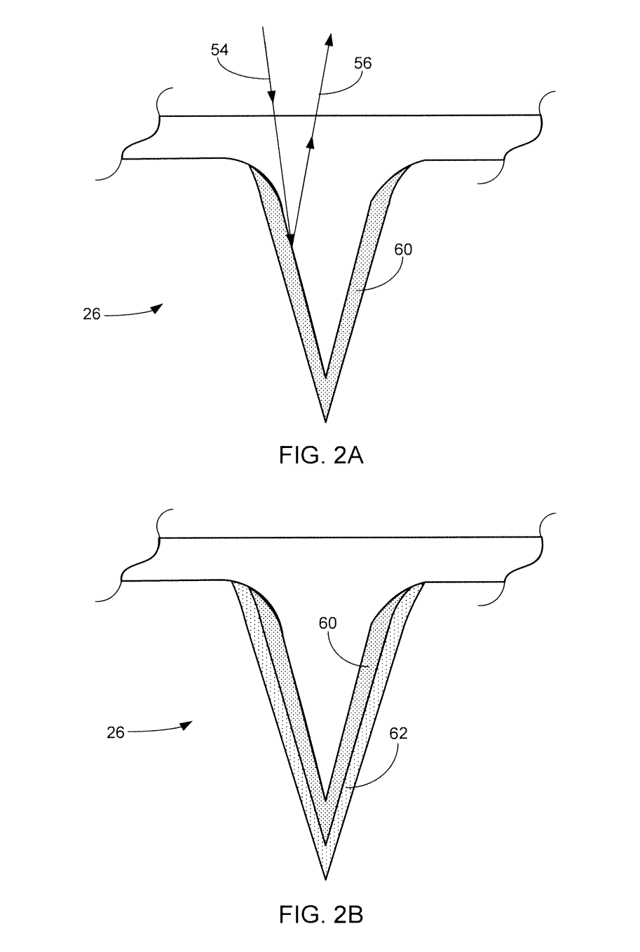

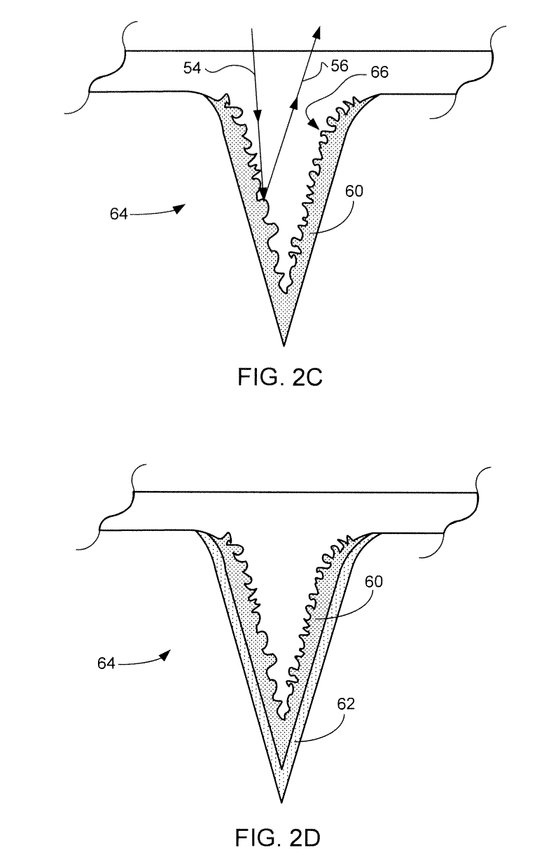

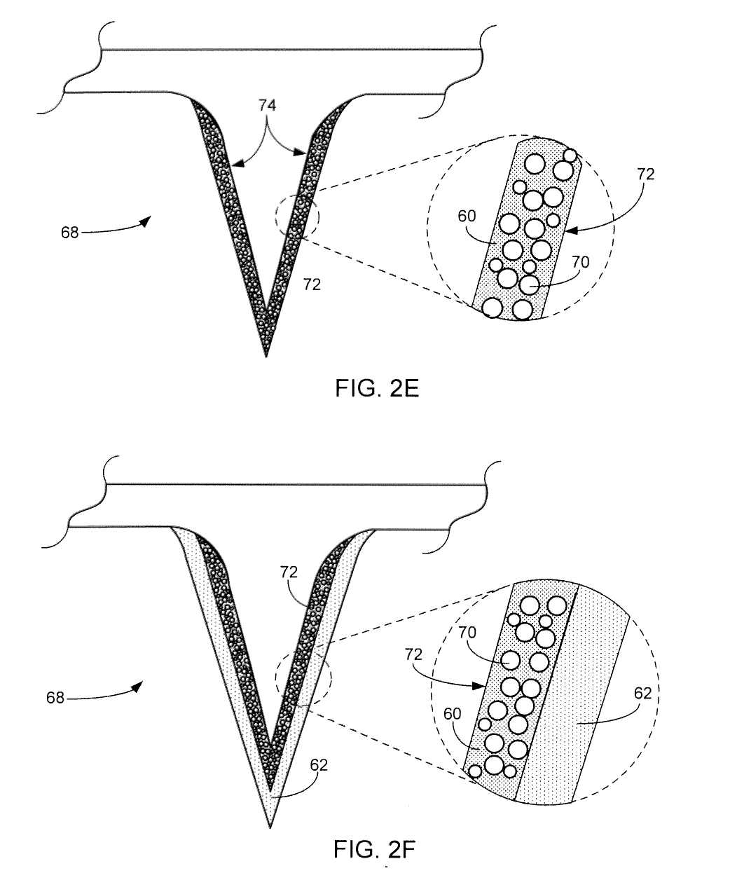

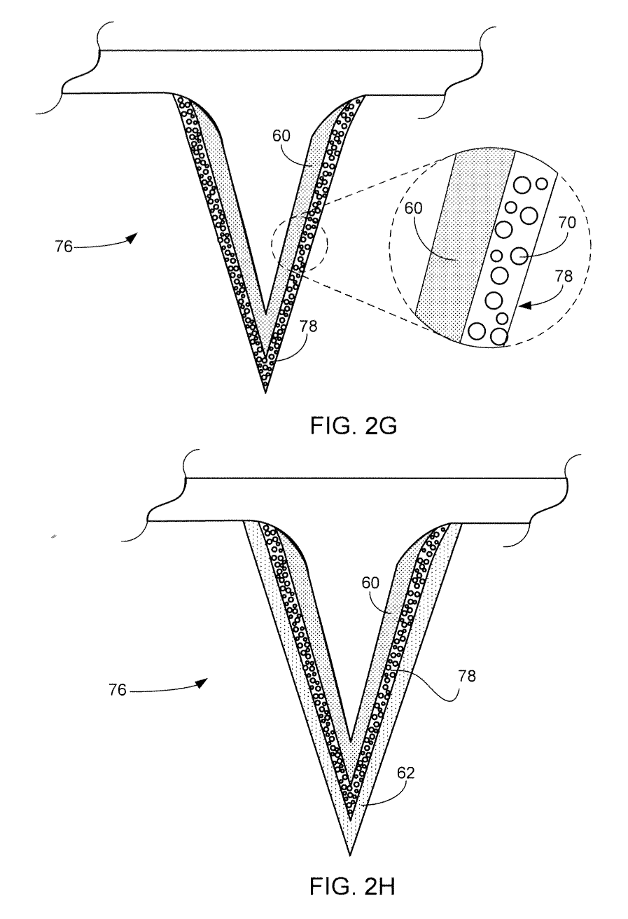

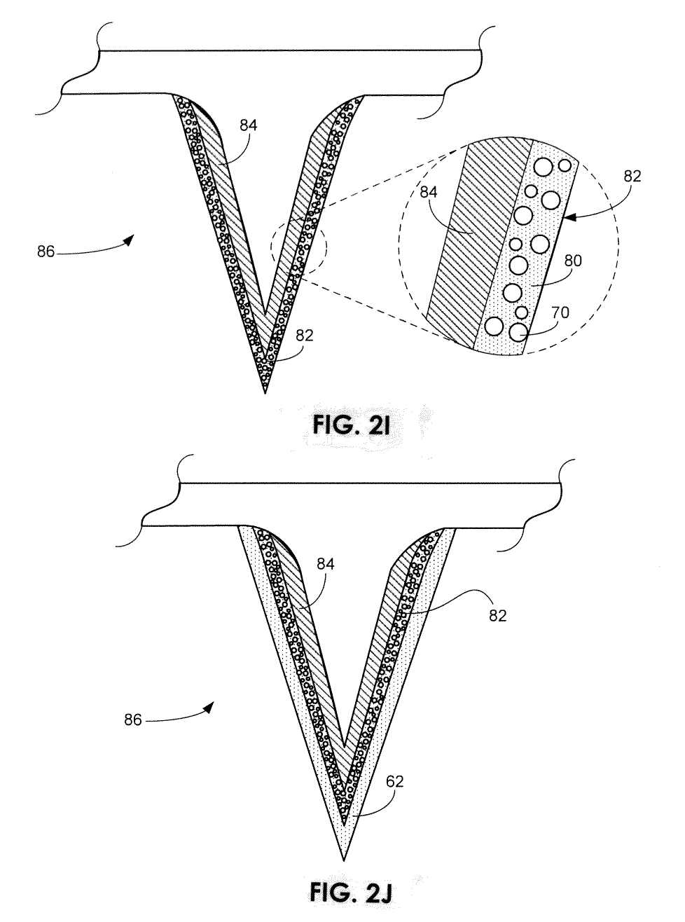

The one or more microneedles 26 can be coated with a chemical sensing material (not shown) that either changes its color or fluoresces or changes its fluorescence characteristics when in contact with one or more specific chemical species. The chemical sensing material may be optically transparent, reflective, opaque, or scattering. Different chemical sensing materials are discussed later with regard to FIGS. 2A-2K.

The microneedle array 22 may be configured to be placed in proximity or contact with a test subject's skin 32. In FIG. 1A, the microneedle 26 is shown in an inactivated state positioned retracted within the microneedle array 22 and not in contact with the skin 32. In some embodiments, the microneedle array 22 may be sealed on at least a test-subject-facing side by a protective film 34. Other embodiments can include a protective film on multiple surfaces (for example on the top and bottom surfaces) of the microneedle array 22 in order to seal the one or more microneedles from interaction with the external environment and/or subject, and in general to help maintain a sterile, dry environment for the one or more coated microneedles 26, prior to use. Some embodiments may also include a desiccant layer (not shown) on protective film 34 to provide a dry environment for the one or more coated microneedles 26. If a protective film is used on the base 30 side of the microneedle array 22, then the protective film is preferably transparent or translucent. Non-limiting examples for a protective film 34 include polyvinylidene fluoride, polyvinyl chloride, polyvinylidene chloride, polypropylene, polyethylene terepthalate, polyethylene napthenate, ethylene-vinyl acetate copolymer, and low density polyethylene. The microneedle array 22 may be a removable and replaceable subassembly which the biomedical monitor 20 is configured to receive.

The biomedical monitor 20 also has at least one actuator 36 configured to move the one or more microneedles 26 from the inactive position illustrated in FIG. 1A to the activated or engaged position illustrated in FIG. 1B, as well as positions in-between in some embodiments. Depending on the embodiment, a single actuator 36 may be provided and moveable relative to the one or more microneedles 26 such that the one or more microneedles 26 may be engaged one at a time by the single actuator 36. In other embodiments, multiple actuators 36 may be provided, each of the multiple actuators 36 corresponding to one of multiple microneedles 26.

The actuator 36 schematically illustrated in the embodiment of FIG. 1A has a transparent actuator substrate 38 with a transparent depressor 40. In other embodiments, the actuator substrate 38 and/or the depressor 40 could be translucent. The actuator 36 is configured so that the transparent depressor 40 may be moved into contact with the microneedle base 30, pushing the microneedle 26 from the inactive position of FIG. 1A to the activated position of FIG. 1B. The actuator 36 of FIG. 1A is just one example of an actuator and those skilled in the art are aware of many other ways to actuate or engage a microneedle. As just one alternate example, the actuator could be integrated with the microneedle restoring springs 28, removing the need for a depressor to contact the microneedle base. Depending on the actuator embodiment, the actuator could be moved based on an applied mechanical force (for example, from an electro-mechanical device), a piezoelectric force, an electrostatic force, or a magnetic force. The actuator 36 may optionally be coupled to a processor 40 which can be configured to control the actuation (on/off) and/or the degree of activation for the one or more microneedles 26.

The biomedical monitor 20 also has an optical system 44 for capturing images of the one or more microneedles 26. The optical system 44 may include one or more light sources 46, an image sensor 48, and optics 50 for focusing an image of the microneedle 26 onto the image sensor 48. In this embodiment, the use of an off-axis light source 46 allows diffuse light reflected from the sensing material coating the microneedles 26 to be captured by the image sensor 48 which is located directly above the sensing material. Other embodiments may have different light source and/or image sensor locations. The symmetrical illumination made possible by the multiple light sources 46 in this embodiment also results in a reduction of shadowing in the microneedle images captured by the image sensor 48. Other embodiments may use different numbers and/or locations of light sources, however.

The optical system 44 may also optionally include obstructions 52 which function to restrict certain angles of illumination and reduce specular reflections from the top surface of the microneedles.

FIG. 1C shows the activated or engaged state of the monitor with the one or more light sources 46 turned on to illuminate the microneedle 26. In some embodiments, it may be desirable to illuminate the microneedle 26 prior to activating the microneedle 26 in order to scatter light from the microneedle 26 that can be captured by the image sensor 48 for a baseline image of the microneedle 26 before insertion into the subject's skin 32. In still other embodiments, it may be desirable to capture images of the microneedle 26 as it is inserted into and/or withdrawn from the skin 32. In further embodiments, it may be desirable to capture images of the microneedle 26 after the microneedle 26 is withdrawn from the skin 32, as illustrated in FIG. 1D. Once the microneedle 26 has sampled the appropriate body fluid within the skin, sufficient time must elapse such that sensing material integral to microneedle 26 undergoes enough of a color change to result in an accurate measurement. Such a waiting time can be from about one second to two minutes, although lesser or longer times may be used. Microneedle 26 should remain inserted in the subject for sufficient time such that the sensing material coated on the microneedle is imbibed with the appropriate body fluid. Through the use of specific image processing algorithms, features such as the penetration depth of the microneedle, the wetting of the sensing material coated on the tip of microneedle 26, and the color change or fluorescence change can be ascertained.

After the microneedle 26 penetrates the subject, the sensing material (not shown) coated on the microneedle 26 undergoes a change in color or exhibits fluorescence which is sampled using the one or more light beams 54 emanating from the one or more light sources 46. As non-limiting examples, light source 46 could be an incandescent source with collimation optics, a light emitting diode, or a laser diode. The spectral requirements for optics 50 will depend on the wavelength required to monitor absorption of the colorant reagent or excite fluorescence in the sensing material coated on the microneedle 26. Signal beam 56 emanating from the sensing material coated on the microneedle 26 includes information regarding the color change of the sensing material, and is focused by optics 50 to form an image of the sensing material on the imaging sensor 48. The imaging sensor 48 may be made selective to the optical absorption or fluorescence wavelengths of the sensing material coated on the microneedles 26. Those skilled in the art will recognize that the exemplary optical path illustrated in FIGS. 1C-1D is just one example of a suitable optical path for capturing images of the microneedle 26. Other embodiments may have fewer, more, and/or different optic path elements such as reflectors, beam splitters, other lenses, etc. Furthermore, in other embodiments, the optic path may follow different trajectories. In another embodiment, actuator 36, substrate 40, and depressor 40 may be mechanically attached to optical system 44. In this case, The optical system 44 would be actuated along with actuator 46 during the activated stage.

The image sensor 48 is coupled to the computing device 42. The image sensor 48 provides image-based output 58 to the processor. Suitable non-limiting examples for an image sensor 48 include a charged coupled device (CCD) image sensor and a complementary metal oxide semiconductor (CMOS) image sensor. Image processing techniques, as will be described later, are employed to intelligently assess the image and modify it to eliminate spatial regions that are determined to be non-representative of good data. Image processing and data manipulation may be performed by computing device 42 to determine a concentration of one or more chemicals being monitored. The determined concentration may be an actual concentration or a number representative of or proportional to the concentration of the chemical being monitored.

The computing device 42 may include a central processing unit (CPU), controller or processor, a memory, and an interface system which are coupled together by a bus or other link, although other numbers and types of each of the components and other configurations and locations for the components can be used. The processor in the computing device 42 may execute a program of stored instructions for one or more aspects of the methods and systems as described herein, including for biomedical monitoring, although the processor could execute other types of programmed instructions. The memory may store these programmed instructions for one or more aspects of the methods and systems as described herein, including methods for biomedical monitoring, although some or all of the programmed instructions could be stored and/or executed elsewhere. A variety of different types of memory storage devices, such as a random access memory (RAM) or a read only memory (ROM) in the system or a floppy disk, hard disk, CD ROM, DVD ROM, or other non-transitory computer readable medium which is read from and/or written to by a magnetic, optical, or other reading and/or writing system that is coupled to the processor, may be used for the memory. The interface system may include one or more of a computer keyboard, a computer mouse, and a computer display screen (such as a CRT or LCD screen), although other types and numbers of interface devices may be used.

Although some embodiments of computing devices 42 for use in the biomedical monitor 20 have been discussed herein for exemplary purposes, many variations of the specific hardware and software used to implement the computing device 42 are possible, as will be appreciated by those skilled in the relevant art(s). Furthermore, the computing device 42 of the biomedical monitor 20 may be conveniently implemented using one or more general purpose computer systems, microprocessors, digital signal processors, micro-controllers, application specific integrated circuits (ASICs), programmable logic devices (PLDs), field programmable logic devices (FPLDs), field programmable gate arrays (FPGAs) and the like, programmed according to the teachings as described and illustrated herein, as will be appreciated by those skilled in the computer, software and networking arts.

In addition, two or more computing systems or devices may be substituted for the computing device 42. Accordingly, principles and advantages of distributed processing, such as redundancy, replication, and the like, also can be implemented, as desired, to increase the robustness and performance of the biomedical monitor 20. The computing device 42 may also be implemented on a computer system or systems that extend across any network environment using any suitable interface mechanisms and communications technologies including, for example telecommunications in any suitable form (e.g., voice, modem, and the like), Public Switched Telephone Network (PSTNs), Packet Data Networks (PDNs), the Internet, intranets, a combination thereof, and the like.

The computing device 42 can further be configured to store data (remotely and/or locally) corresponding to the biomedical characteristic being measured, together with subject information, date, and time, all of which may comprise an electronic medical record. The electronic medical record can be generated automatically and can be recalled and displayed on the biomedical monitor 20. The electronic medical record can also be transmitted automatically or on command using wireless or other techniques well known in the information technology arts.

FIGS. 2A-2K schematically illustrate embodiments of sensing materials coated on microneedles, in cross-sectional views, for use with a biomedical monitor. As shown in FIG. 2A, the microneedle may be illuminated by light beam 54. A signal beam 56 may result from diffuse reflection of the light beam 54 of and/or from fluorescence excited by the incident light beam 54. The microneedle 26 may be coated with a chemical sensing material 60 that either changes its color or fluoresces or changes its fluorescence characteristics when in contact with a specific chemical specie. In some embodiments, the chemical sensing material 60 may be incorporated into a porous matrix capable of imbibing body fluid when inserted into the dermis. Chemical sensing material 60 for blood glucose monitoring may use a large number of known glucose sensitive chemicals, such as, but not limited to glucose oxidase, glucose dehydrogenase, hexokinase-glucokinase, rhenium bipyridine, boronic acid containing fluorophores, NBD-fluorophores, Europium tetracycline, and combinations, or any other materials that exhibit the desired chemical and optical response. A preferred chemical sensing material includes glucose oxidase, peroxidase, and an oxidizable colorant or colorant precursor. The colorant or colorant precursors are preferably non-toxic, non-carcinogenic, and non-mutagenic. Suitable non-limiting examples of colorants include oxidizable color-change dyes such as 4-aminoantipyrine, chromotropic acid, and the like.

A particularly preferred colorant for glucose testing includes potassium iodide and amylose. Potassium iodide is oxidized to produce polyiodide ion that in the presence of amylose forms a complex that is a very strong optical absorber having a blue-violet color. Amylose is a polysaccharide and a component of vegetable starches. Vegetable starch may in fact be used directly in the chemical sensing material 60, the starch also providing function as a binder and film-forming agent. Other strongly colored tri-iodide ion-host systems include tri-iodide plus polyvinyl acetate, polyvinyl alcohol, polyvinyl pyrrolidone, nylon, cellulose, chitosan or combinations of these host materials. Other poly-atom iodide ions exist and can also form strongly colored complexes in the above host systems.

It should be apparent to those skilled in the chemical arts that these examples of chemical sensing materials are merely illustrative of broader families of chemicals. It will be apparent to those skilled in the chemical arts that the example materials may be modified while still performing the same or similar function of providing or facilitating a spectral response in the presence of a target chemical or chemical compound. All such modifications and equivalents to the listed chemical sensing media as well as alternates for other target media besides glucose are intended to be included in this disclosure. In some cases, the reagent or fluorophore may need to be incorporated into a polymeric matrix in order to achieve coatability, adhesion, or chemical stability. Other reagents or fluorophores may be used to monitor cholesterol, HDL cholesterol, LDL cholesterol, alcohol, estrogen-progesterone, cortisol, and other physiological chemicals of interest.

During the wetting of the chemical sensing material 60 with body fluid, the mass flow into the chemical sensing material will tend to mitigate potential diffusion of components of the chemical sensing material into the subject. After filling, however, slow diffusion from the chemical sensing material 60 to the subject may occur. Therefore, in some embodiments, such as the microneedle 26 illustrated in FIG. 2B, it may be desirable to include a semi-permeable membrane overlay 62 to prevent or mitigate diffusion of certain species from chemical sensing material 60 to the subject. Optimally, the membrane 62 freely passes water and the analyte of interest, for example, glucose. It is also sometimes desirable that the membrane 62 is oxygen permeable. The membrane layer 62 can also function to improve the mechanical integrity of the coated chemical sensing material 60. In some cases, membrane layer 62 may contain some constituents of the sensing chemistry. For example, a colorant precursor such as potassium iodide may be included as part of membrane layer 62 so that when body fluid is imbibed into 62, the colorant precursor is dissolved and carried to sensing layer 60 along with the chemical specie being monitored.

Although the tissues within the dermis are diffusely reflective and can function to reflect light incident on the microneedle back to the image sensor, the amount of the light reaching the image sensor may be enhanced by utilizing a roughened microneedle 64 as illustrated in FIG. 2C. The microneedle 64 may be roughened, for example, through the use of etching techniques known to those skilled in the art. A chemical sensing material 60 may be coated on the roughened microneedle 64. The roughened surface 66 of microneedle 64 will tend to increase the amount of incident light beam 54 which is reflected back towards the image sensor as signal beam 56. Optionally, a semi-permeable membrane overlay 62, as discussed above, may be included on the roughened microneedle 64 as illustrated in FIG. 2D.

The amount of light reaching the image sensor may alternatively be enhanced with the inclusion of micro-particulate diffuse reflection/scattering particles with the chemical sensing material. For example, the microneedle 68 shown in FIG. 2E has micro-particulate diffuse particles 70 distributed throughout the chemical sensing material 60 as part of a film 72. The micro-particulate diffuse particles 70 may be high refractive index materials (for example, with a refractive index of about 2.5 or higher) or they may be low refractive index materials (for example with a refractive index of about 1.35 or lower), although higher or lower refractive index materials may be used in some embodiments for the micro-particulate diffuse particles 70. Non-limiting examples of micro-particulate diffuse particles having a high index of refraction include TiO₂, ZrO₂, HfO₂, Ta₂O₅, Al₂O₃, ZnO, SnO₂, CaCO₃ and the like. Though these materials are transparent throughout the visible, other high index inorganics having some visible absorption are also of use since, as will be described later, it is preferred that the spectral measurements undertaken to ascertain the biomedical characteristic of interest involve ratios of intensities at different wavelengths. Exemplary colored high-index inorganic materials that can be used as micro-particulate aids to diffuse reflectance include ZnSe, ZnS, ZnSe₍₁₋ₓ₎Sₓ, GaP, and the like. Alternatively, the micro-particulate diffuse particles 70 can be of very low effective refractive index, such as readily available glass or polymer micro-balloons.

The chemical sensing material 60 may include the specific analyte selective agent or agents, typically enzymes, the oxidizable colorant system or fluorescent material, and film-forming binders. Binding materials of use may include natural or synthetic polymers such as latex, starch, polyvinyl alcohol, polyvinylpyrrolidone, ethyl cellulose, methylvinylether/maleic anhydride copolymer, and acrylic, vinyl acetate, styrene and butadiene homo- and copolymers and the like. It is preferred that the film 72 is well-adhered to the microneedle at interface 74, and that it exhibits good cohesion. It is also preferred that the film 72 exhibits an openly porous microstructure. The openly porous structure will facilitate a rapid filling with body fluid by capillary forces when microneedle 68 is inserted into the subject. The openly porous structure can be achieved using the micro-particulate diffuse particles disclosed above together with appropriate amounts of binder. Increasing the amount of binder tends to result in more mechanical strength at the expense of fluid retention speed, while reducing the amount of binder tends to increase fluid retention speed at the expense of mechanical strength.

In alternate embodiments, porous metal oxide or mixed metal oxide films (comprising the chemical sensing material) may be prepared by the sol-gel method, well known in the art. Alternatively, polymeric materials can form porous film coatings by use of the well-known mixed-solvent techniques for producing porous polymer films. In a related approach, immiscible mixtures of polymers can form films having segregated polymer phases that can form porous films by dissolving away one of the polymer phases. Cellulose systems are particularly useful for forming porous polymer films. For example, ethyl cellulose and hydroxypropylcellulose or hydroxypropyl methylcellulose constitute preferred mixed phase systems. Preferred mixed solvent systems for ethyl cellulose include water and propanol, water and ethanol, acetone and propanol, and the like. Cellulose acetate or cellulose acetate-butyrate used with a mixed acetone/water solvent or pore formers such as magnesium perchlorate, polyethylene glycol also are preferred porous film-forming systems. Microfibrous films having a paper-like microstructure are also useful porous films. Once-filled with body fluid, for example interstitial fluid found in the dermis, the coated microneedle can optionally be retracted and imaging of the imbibed coated microneedle undergoing reaction can be continued as described above. To achieve the desired rapid filling, it is preferred that the porous film 72 which includes chemical sensing material 60 have a means to vent the air that will be initially contained within it. This can be accomplished at the upper portions of the film 72 that are positioned in a dry zone above the location of the skin penetration.

Optionally, a semi-permeable membrane overlay 62, as discussed above, may be included on the microneedle 68 as illustrated in FIG. 2F.

In the microneedle 76 embodiment illustrated in FIG. 2G, the micro-particulate diffuse particles 70 are not dispersed throughout the chemical sensing material 60. Instead, the chemical sensing material 60 is in its own distinct layer, while the micro-particulate diffuse particles 70 are divided into a separate scattering film/layer 78. In some embodiments, it remains desirable that the separate scattering film 78 have an openly porous microstructure. The micro-particulate diffuse particles 70 may be held in place by a binder. Additionally, the interfaces between layers should exhibit good adhesion.

Optionally, a semi-permeable membrane overlay 62, as discussed above, may be included on the microneedle 76 as illustrated in FIG. 2H.

In the embodiments of FIGS. 2G and 2H, the chemical sensing material 60 resides in its own sensing layer, the chemical sensing material including both the analyte-selective species and the indicator colorant materials. It is sometimes desirable, as in the case of glucose oxidase/peroxidase-induced oxidation of a colorant for glucose detection, to place the analyte-selective species 80 (such as enzymes) and the micro-particulate diffuse particles 70 in a combined film/layer 82 with the color-forming components in layer 84 as shown in the microneedle 86 embodied in FIG. 2J. This is because oxygen may be a reactant in such a system and the reaction will proceed faster if the enzymes are located in the porous outer layer 82. The need for oxygen in such reactions provides motivation to withdraw the microneedle 86 soon after its insertion into the subject.

Optionally, a semi-permeable membrane overlay 62, as discussed above, may be included on the microneedle 86 as illustrated in FIG. 2K.

Combinations of one or more configurations as shown in FIGS. 2A-2K may also be useful.

Although the analyte-selective species and the indicator materials may be sufficiently immobilized by physical sequestering, it is sometimes desirable to use chemical techniques. It is known in the art that enzymes and dyes may be immobilized at surfaces of both inorganic and polymeric materials. For example, benzoate, carboxylate, sulfonate, salicylate and phosphonate compounds are useful in binding dyes to inorganic oxides as taught in Electrochemistry of Nanomaterials by G. Hodes p. 148 and in U.S. Patent Application Publication No. 2008/0128286 to Wu et al. paragraph 34, both of which are hereby incorporated by reference in their entirety. “Comparison of techniques for enzyme immobilization on silicon supports” by Aravind Subramanian et al. published in Enzyme Microb. Technology, 1999, 24, 26-34, also incorporated herein by reference, teaches techniques for anchoring enzymes such as glucose oxidase to silicon/silicon dioxide surfaces. N. Gupta et al in Journal of Scientific and Industrial Research, Vol 65, 2006, p. 535, further incorporated herein by reference, teaches the use of a number of immobilizing matrices for the enzyme glucose oxidase. These include tetrathiofulvalene with tetracyanoquinodimethane, polypyrrole, poly(ethylene-vinyl alcohol), polyphenol, polyurethane, and polyethylene-g-acrylic acid Immobilization of enzymes in hydrogel matrices of sol-gel oxide films, e.g. SiO₂ gel is also well known. For polymeric porous media, surface functionalization with reactive groups, epoxy or amino groups, for example, is a well-known technique for immobilization of enzymes.

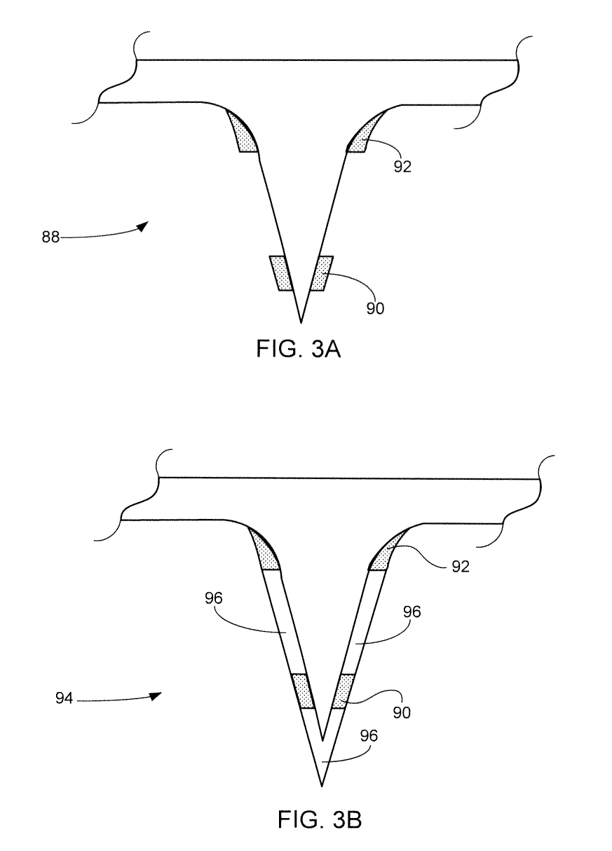

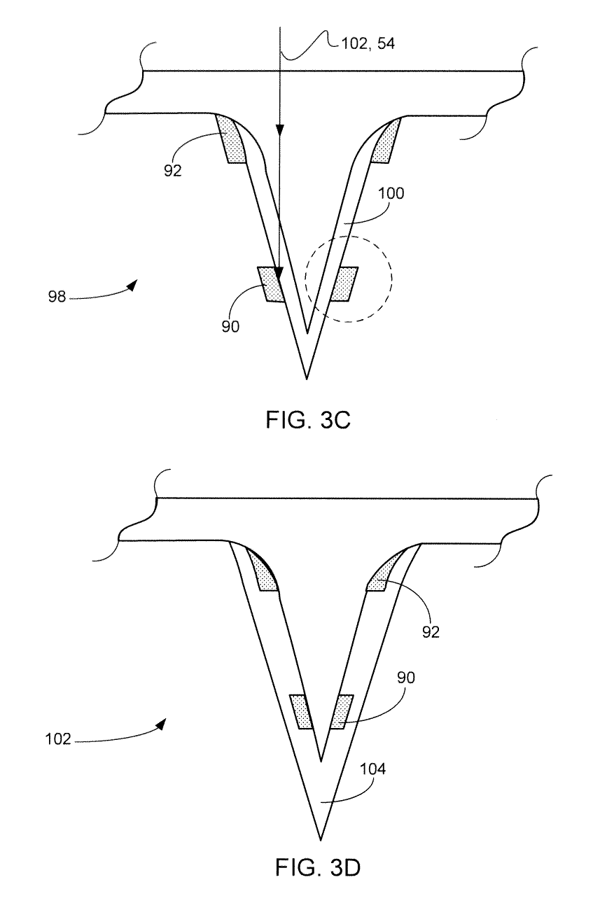

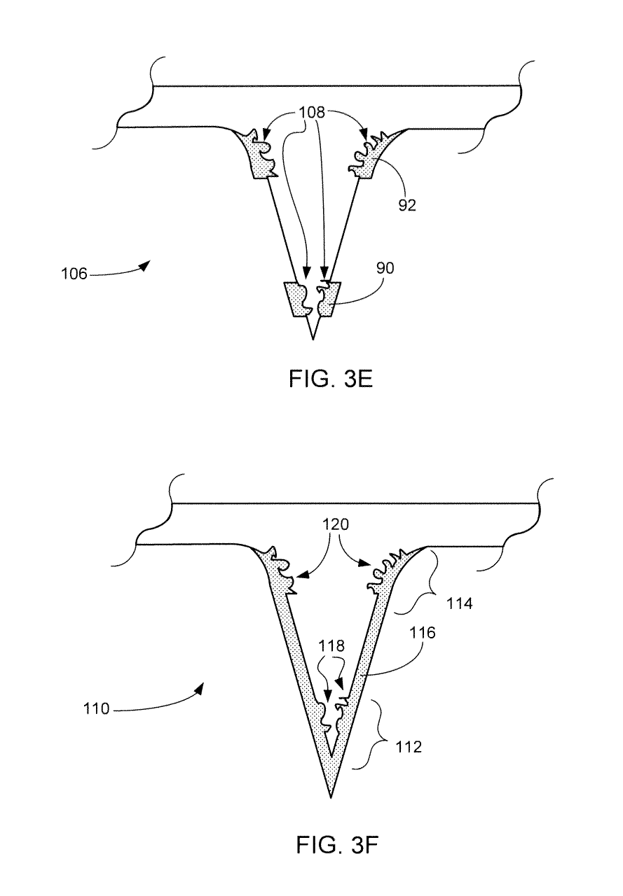

The microneedles in the microneedle array do not need to be limited to having a single sensing region. For example, FIGS. 3A-3F schematically illustrate embodiments of multiple sensing regions coated on microneedles, in cross-sectional views, for use with a biomedical monitor. FIG. 3A shows the side cross-sectional view of one embodiment of a microneedle 88 that includes multiple regions of chemical sensing material 90 and 92. Each of the multiple regions of chemical sensing material 90, 92 may be configured to react with the same analyte or different analytes. The spatial image processing methods (to be described in more detail further on) performed on images of the microneedle captured by the biomedical monitor's image sensor may be configured to separately identify and analyze the different regions of chemical sensing material 90, 92. This potentially allows for more tests to be completed within a smaller area. Multiple sensing regions at different heights on the microneedle could be monitored by the biomedical monitor to determine an insertion depth of the microneedle corresponding to color changes in sensing regions at different heights along the microneedle. Multiple sensing regions at different heights could also be used to compare analyte concentrations at different test depths.

FIG. 3B shows the side cross-sectional view of another embodiment of a microneedle 94 that includes multiple regions of chemical sensing material 90 and 92 combined with a capillary/porous layer 96. Suitable non-limiting examples of capillary layers/films have been discussed above. The capillary layer 96 may speed up the drawing of body fluid for mixture with the regions of chemical sensing material 90, 92, and may also make it possible to insert the microneedle 94 less far into a test subject's skin since the sampled body fluid may be drawn up into the film 96 above the skin. As with the above embodiments, each of the multiple regions of chemical sensing material 90, 92 may be configured to react with the same analyte or different analytes.

FIG. 3C shows the side cross-sectional view of another embodiment of a microneedle 98 that includes multiple regions of chemical sensing material 90 and 92 placed on the surface of a capillary/porous layer 100. Suitable non-limiting examples of capillary layers/films have been discussed above. Incident light 54 propagates through the capillary layer 100 and into the regions of chemical sensing material 90, 92. A fraction of the light 54 propagating in the capillary layer 100 will be transmitted into the regions of chemical sensing material 90, 92 depending upon the indices of refraction of the capillary layer 100 and the regions of chemical sensing material 90, 92.

FIG. 3D shows the side cross-sectional view of a further embodiment of a microneedle 102 that includes a capillary/porous layer 104 over the multiple regions of chemical sensing material 90 and 92. Suitable non-limiting examples of capillary layers/films have been discussed above. In such an embodiment, the capillary layer 104 may protect the multiple regions of chemical sensing material 90, 92 against abrasion or removal while drawing body fluid into contact with the multiple regions of chemical sensing areas. Such a capillary layer must have one or more regions that are permeable to the analyte(s) in question, for example glucose, to enable monitoring by the biomedical monitor.

FIG. 3E shows the side cross-sectional view of another embodiment of a microneedle 106 that includes multiple regions of chemical sensing material 90 and 92, each coated onto one or more roughened surfaces 108 of the microneedle 106. As described above, the roughened surfaces 108 can help increase the amount of light which is reflected back to the image sensor of the biomedical monitor. Although the roughened surfaces in the embodiment of FIG. 3E are separate for each region of chemical sensing material 90, 92, in other embodiments, the entire surface of the microneedle could be roughened even if there were multiple regions of chemical sensing material 90, 92. As with the above embodiments, each of the multiple regions of chemical sensing material 90, 92 may be configured to react with the same analyte or different analytes.

FIG. 3F shows the side cross-sectional view of another embodiment of a microneedle 110 that includes multiple effective regions of chemical sensing material 112, 114 created from a single coating of a chemical sensing material 116 over multiple roughened surfaces 118, 120 of the microneedle 110. As described above, the roughened surfaces 108 can help increase the amount of light which is reflected back to the image sensor of the biomedical monitor. If the multiple roughened surfaces 118, 120 are at different heights, the multiple effective sensing regions 112, 114 could be monitored by the biomedical monitor to determine an insertion depth of the microneedle corresponding to color changes in sensing regions at different heights along the microneedle. Similarly, the multiple effective sensing regions 112, 114 at different heights could also be used to compare analyte concentrations at different test depths.

Optionally, a semi-permeable membrane overlay, as discussed above, may be included on the microneedles as illustrated in FIGS. 3A-3F.

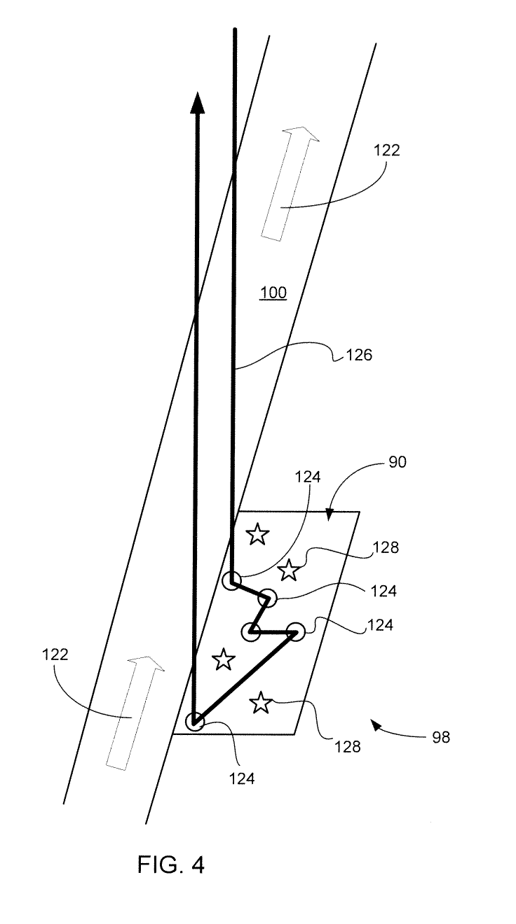

FIG. 4 shows the highlighted region of FIG. 3C providing an expanded view of the microneedle 98. Fluid flow 122 from capillary action in the capillary layer 100 causes interstitial fluid containing analytes to pass along the chemical sensing region 90. The capillary layer 100 can include a number of porous materials, including, for example, porous silicon, porous silicon dioxide, porous titania, paper, silk, porous cellulose acetate, and a variety of other materials as disclosed earlier in the detailed description. Preferably, the material selected for the capillary layer 100 exhibits high capillarity, is hydrophilic, is transmissive to light at the wavelength or wavelengths of interest, and is a stable environment for the chemistries that occur in the region of chemical sensing material 90. Although it is preferred to have the capillary layer 100 be a hydrophilic material, in some embodiments it may be possible to use a hydrophobic material.

As discussed above, it is also possible in other embodiments to position the capillary layer so that it is disposed outside of the region of chemical sensing material 90. Capillary flow can be quite significant causing the displacement of interstitial fluid to the region of chemical sensing material 90 within seconds of placing at least a portion of the microneedle 98 beneath the skin surface. Diffusion of the reagent species within the region of chemical sensing material 90 and into the capillary layer 100 is opposed to this flow and thereby contamination of the patient by the backflow of the reagent species is precluded. One or more scattering centers 124 are illustrated within the region of chemical sensing material 90. Such scattering centers 124 redirect the path of an optical ray 126 from its normal straight line path into a different direction. Multiple scattering events can cause the path of the optical ray 126 to come back upon its original direction. Thus, through the use of such scattering centers 126, the light from a light source (not shown) can be brought back up through the microneedle and made available for image detection.

The scattering centers 124 may take a variety of material forms, for example, but not limited to titanium dioxide and silicon dioxide. Additionally, porous silicon or titanium dioxide are materials that exhibit capillary action and so could act as either the capillary layer 100 or the scattering centers 124. Other materials such as polymers, organic compounds, and inorganic compounds are also candidate materials, as discussed earlier in the detailed description. One guideline for material suitability for scattering centers is that they scatter light in the wavelength of interest and do not interfere with the chemical reactions described below that result in detection of the analyte. Although FIG. 4 shows both scattering centers 124 and reagent centers 128 in proximity to each other, there may be non-reagent regions where only scattering centers 65 are found. Such non-reagent regions would serve to scatter or reflect incoming light 126 from the source back to a suitable detector. In this manner non-reagent regions could provide a mechanism to measure the intensity of the light 126 incident from a source in each individual microneedle 98. By being able to determine light intensity, a system may be configured to compensate for variations in the intensity of light 126 over time, or the variation of light throughput across numerous individual microneedles.

Reagent centers 128 include those specific molecules or materials that respond with a change in some optical property to the presence of the analyte. For glucose detection, there are many chemistries known that exhibit change in some optical property due to the presence of the glucose molecule, some of which were described previously in this disclosure. Following is a more detailed description of sensing material chemistries and optical properties that can be used in microneedle arrays. One such optical property change is a color change in which a dye molecule or other species undergoes a shift in its absorption or reflectance spectrum as a result of reaction with an analyte (for example, glucose) or a product of a reaction of the analyte with some other molecule or species that reacts specifically with the analyte. Thus generally the chemistries are divided into analyte sensing components that produce a reaction product and analyte indicator components that react with the reaction product to produce an optical change. One example of an analyte sensing component is the enzyme glucose oxidase. Dyes, nano-sized metal particles (e.g. gold), and a variety of inorganic and organic materials have demonstrated the ability for reflective or transmissive color change in the presence of a specific analyte or analyte reaction product.

Another optical property to be considered is luminescence. Those skilled in the art will appreciate that luminescence includes both fluorescent and phosphorescent light emission mechanisms. Reagent centers 128 can indicate the presence of the analyte by the production of a luminescent compound, or by producing a change in a luminescent compound property, such as emission wavelength, emission lifetime, emission polarity, and others. The specificity of the reagent centers 128 is largely determined by the chemical binding properties of the analyte to the reagent center 128 molecule or molecules. Examples of fluorescent-based reagent centers 128 include, but are not limited to synthetic boronic acid derivatives and as has been already mentioned, the enzyme glucose oxidase. Glucose oxidase (GOₓ) has been widely employed in glucose sensing. GOₓ catalyzes the conversion of D-glucose and oxygen to D-glucono-1,5 lactone and hydrogen peroxide. The detection of oxygen consumption, hydrogen peroxide production, or local pH change has been widely utilized in the development of GOₓ-based glucose sensors as they correlate with the levels of glucose present in a given sample. The simplest strategy employed for the development of a fluorescent glucose sensing system based on GOₓ takes advantage of the intrinsic fluorescence of the biomolecule. GOₓ exhibits an intense fluorescence signal with excitation at wavelengths of 224 nm and 278 nm, and emission at 334 nm.

The use of two or more types of reagent centers 128 enables a multi-analyte microneedle 98 to overcome the limitations of certain detection chemistries described above. Imperfect specificity of reagent center detection chemistry may result in the production of false positive measurements of a particular analyte. For example, certain boronic acid derivatives useful in fluorescent change detection schemes have significant sensitivity to fructose. A combination of reagent centers 128 with differing sensitivity and specificity to specific analytes could provide a superior measurement of the analyte using a matrix algebra approach to the analysis data.

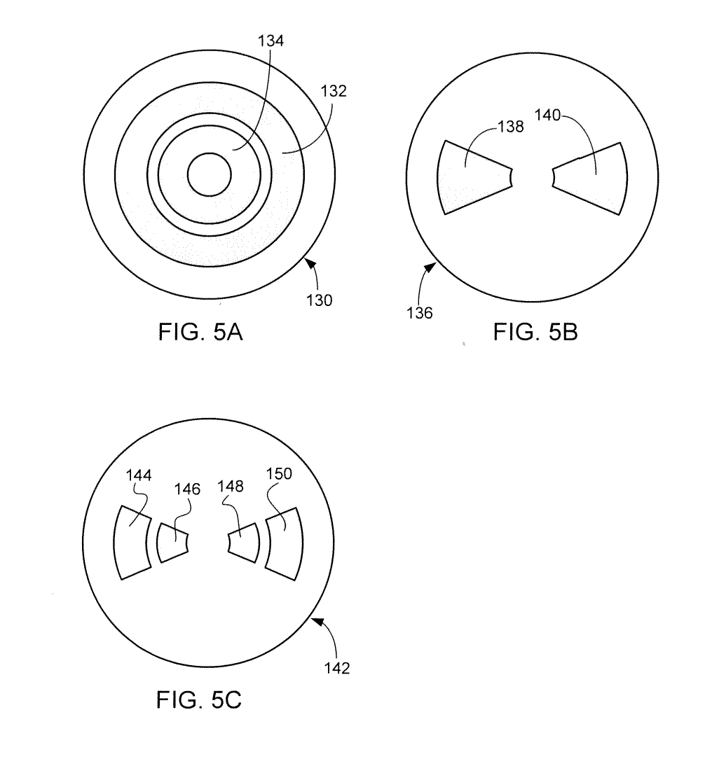

Distribution of the multiple regions of chemical sensing materials on a microneedle may be performed in a number of ways. FIGS. 5A-5C schematically illustrate non-limiting examples of different spatial arrangements for multiple regions of chemical sensing materials from a top view (similar to what could be viewed by the image sensor of a biomedical monitor). In the microneedle 130 of FIG. 5A, a first region of chemical sensing material 132 and a second region of chemical sensing material 134 are shown as annular rings. Though not shown, annular rings could be disposed contiguously on the micro-needle surface. For example, annular regions 132 and 134 can be mutually abutting, sharing a common annular boundary. FIG. 5B illustrates another embodiment of a microneedle 136 having a first region of chemical sensing material 138 and a second region of chemical sensing material 140 disposed radially on the microneedle 136. FIG. 5C illustrates a further embodiment of a microneedle 142 having first, second, third, and fourth regions of chemical sensing materials 144, 146, 148, and 150. In this embodiment, the regions of chemical sensing areas have both annual and radially divided components. As described previously, each of the multiple regions of chemical sensing materials may have the same or different detection chemistries. It should also be understood that although examples have been shown having two or four multiple regions of chemical sensing materials, other embodiments of microneedles may have any number of regions of chemical sensing material.

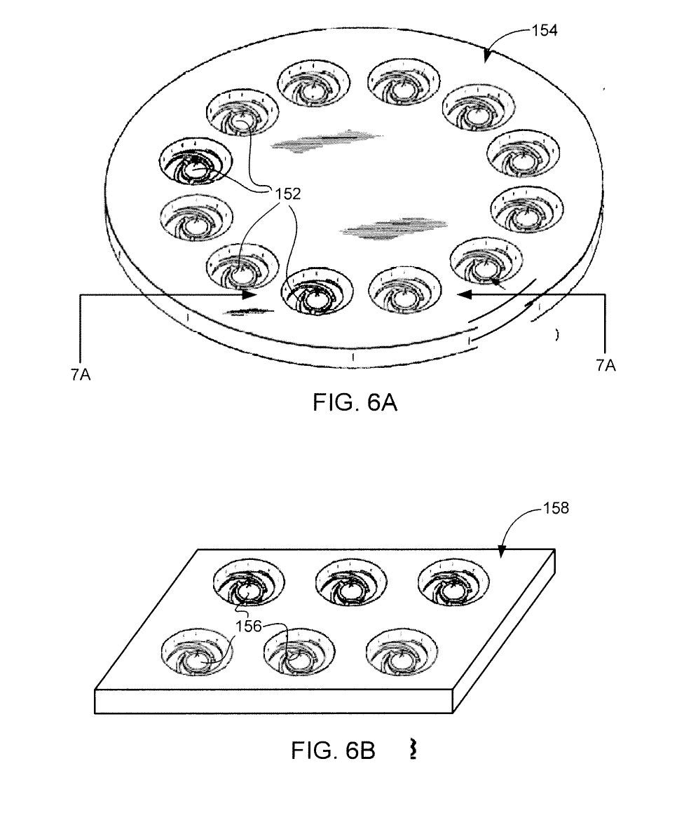

FIGS. 6A and 6B illustrate embodiments of a microneedle array for use with a biomedical monitor in a needle-up view. The needle-up side of the array would typically come into contact or be in close proximity to a test subject's skin in use. In FIG. 6A, the plurality of microneedles 152 in microneedle array 154 are laid out in a rotary array fashion. In FIG. 6B, the plurality of microneedles 156 in microneedle array 158 are laid out in a grid fashion. Those skilled in the art will appreciate that other microneedle array layouts may be used in other embodiments. The microneedle arrays may be a replaceable microneedle array suitable for use with a biomedical monitor as described above. The microneedle arrays may include actuator elements to help engage the microneedles, or the biomedical monitor may include one or more actuators to engage the microneedles of the microneedle array. The replaceable microneedle arrays may be moveable by the biomedical monitor so that different microneedles are aligned with an actuator and the optical system at different times, or the biomedical monitor may be configured to move the actuator and/or optical system to align with different microneedles of the microneedle array.

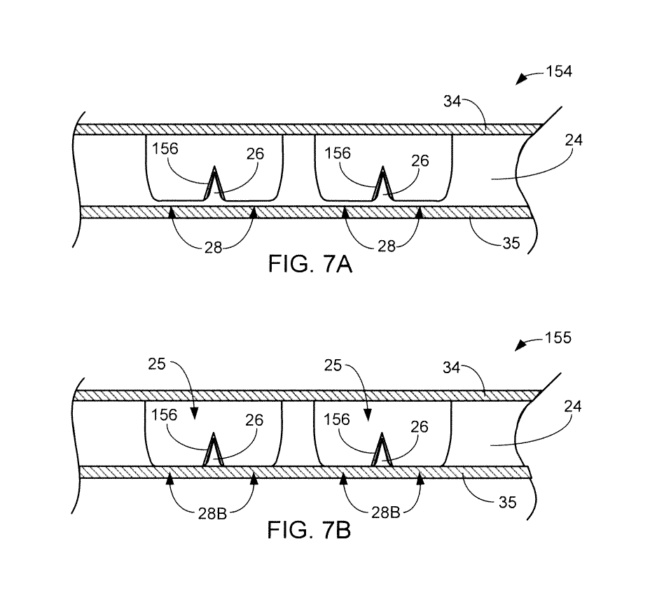

FIG. 7A schematically illustrates a cross-sectional view of a portion of the microneedle array 154 from FIG. 6A taken along cross-section line 7A-7A. In accord with above descriptions of microneedle arrays, the microneedle array 154 may include a substrate 24 which has been micro-machined or precision molded to define one or more microneedles 26 supported by at least one restoring spring element 28. The one or more microneedles 26 should be dimensioned to penetrate the subject's stratum corneum and reach the underlying interstitial fluid or capillary network. The microneedles 26 can be very fine, on the order of 5-50 microns in diameter at the tip, and from 20-2000 microns in height, although smaller or larger diameter and/or height needles may be used in other embodiments. The at least one restoring spring element 28 could be patterned directly out of the substrate 24 material or out of a layer having desirable mechanical properties that has been deposited onto substrate 24. Alternatively, restoring spring 28 may also be patterned out of one or more materials in a multi-material substrate where additional materials have been deposited on or bonded to the substrate 24. For example, an oxidized substrate may be etched to form the one or more microneedles 26 out of silicon and a restoring spring 28 out of either the silicon dioxide layer or a combination of the silicon dioxide layer and the silicon layer. Similarly, using technology such as SOI (silicon-on-insulator), a silicon dioxide microneedle may be etched and the restoring spring be patterned out of the silicon layer. Although not illustrated in this embodiment, other embodiments may include positional sensors on the restoring springs 28 for use in determining the deflection of the microneedle 26. The at least one restoring spring 28 can be patterned in a number of geometries such as a spiral spring, a cantilever structure, or other geometries as long as they provide the freedom of movement that allows microneedle 26 to protrude far enough out of a plane defined by substrate 24 in order to penetrate a subject's skin to a desired depth.

A number of substrate 24 and/or microneedle 26 materials maybe used, e.g. silicon, silicon dioxide, silicon nitride, all commonly used in microfabrication or, in general, dielectrics, plastics, metals, glass, quartz, or sapphire. The microneedle 26 and a base of the microneedle 26 are preferably transparent, but may be translucent in some embodiments. Another option would be to have the bulk material of the microneedle be transparent, while its surface be scattering or translucent. Several fabrication techniques for the one or more microneedles 26 are disclosed in the literature, such as photolithography, reactive ion etching, isotropic etching (e.g. for glass), plastic molding, water jet milling, and others may be used. The one or more microneedles 26 may be solid or hollow. The microneedle 26 cross-sections may be variable or constant, and can take on a variety of cross-sectional shapes, including, but not limited to square, circular, triangular, and grooved. Other embodiments of microneedles 26 may even be corrugated.

The one or more microneedles 26 can be coated with one or more regions of a chemical sensing material 156 that either changes its color or fluoresces or changes its fluorescence characteristics when in contact with one or more specific chemical species as discussed above. The chemical sensing material 156 may be optically transparent, reflective, opaque, or scattering.

In some embodiments, the microneedle array 154 may be sealed on at least a test-subject-facing side by a protective film 34. Other embodiments can also include a protective film 35 on the opposite side of the array 154 in order to seal the one or more microneedles 26 from interaction with the external environment and/or test subject, and in general to help maintain a sterile, dry environment for the one or more coated microneedles 26, prior to use. Some embodiments may also include a desiccant layer (not shown) on one or more of the protective films 34, 35 to provide a dry environment for the one or more coated microneedles 26. If a protective film 35 is used on the base side of the microneedle array 154, then the protective film is preferably transparent or translucent. Non-limiting examples for a protective films 34, 35 include polyvinylidene fluoride, polyvinyl chloride, polyvinylidene chloride, polypropylene, polyethylene terepthalate, polyethylene napthenate, ethylene-vinyl acetate copolymer, and low density polyethylene. The microneedle array 154 may be a removable and replaceable subassembly which the biomedical monitor 20 is configured to receive.

FIG. 7B schematically illustrates a cross-sectional view of another embodiment of a microneedle array 155. The microneedle array 155 has a substrate 24 which has been micro-machined or precision molded to define one or more wells 25. The microneedle array 155 also has a backside film 35 opposite a microneedle facing side of the array and covering the wells 25. The backside film is preferably transparent or translucent. Microneedles 26 are formed on the backside film 35 and aligned within each of the wells 25. The backside film 35 acts as a restoring spring element 28B coupled between each microneedle 26 and the substrate 24 such that each of the plurality of microneedles 26 is held at least partially in an associated well 25. The one or more microneedles 26 should be dimensioned to penetrate the subject's stratum corneum and reach the underlying interstitial fluid or capillary network. The microneedles 26 can be very fine, on the order of 5-50 microns in diameter at the tip, and from 20-2000 microns in height, although smaller or larger diameter and/or height needles may be used in other embodiments.

The one or more microneedles 26 can be coated with one or more regions of a chemical sensing material 156 that either changes its color or fluoresces or changes its fluorescence characteristics when in contact with one or more specific chemical species as discussed above. The chemical sensing material 156 may be optically transparent, reflective, opaque, or scattering.

In some embodiments, the microneedle array 155 may also be sealed on at least a test-subject-facing side by a protective film 34. The microneedle array 155 may be a removable and replaceable subassembly which the biomedical monitor 20 is configured to receive.

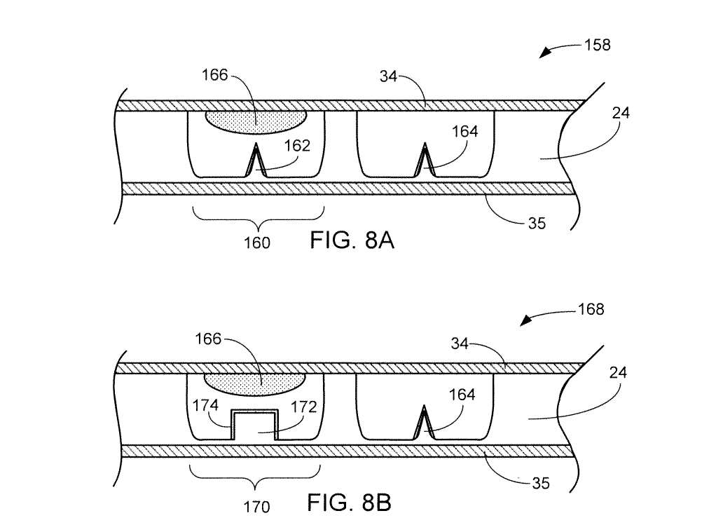

FIG. 8A schematically illustrates a cross-sectional view of a portion of another microneedle array 158 embodiment having a calibration position 160. Each replaceable microneedle array 158 may include one or more calibration positions 160. The coated microneedle 162 at the calibration position 160 can be like the others 164 in the array. However, the protective film 34 that seals the tip end of the microneedle array 158 may include an analyte reference deposit 166 on the microneedle side of the protective film 35 facing the coated microneedle 162 of the calibration position 160. The reference analyte solution 166 may be deposited by a variety of well-known techniques such as ink-jet printing, screen printing, flexographic printing, micropipetting, microdispensing and the like. Preferably, the standard/reference analyte solution should have very low vapor pressure to minimize evaporation. This can be achieved, for example, by using a mixture of solvents that include water and a sufficient amount of glycerol (e.g. 50% or more w/w glycerol). The glycerol/water solution will quickly establish a partial pressure of water vapor in the space within the well (numeral) that encloses coated microneedle 168. In this way the volume of the reference analyte 166 and therefore the analyte concentration will be constant over time. After insertion of a new replaceable coated microneedle array 158, the biomedical monitoring system can actuate the calibration microneedle 162 of the calibration position 160 to measure the reference analyte 166 for calibration purposes.

Alternatively, FIG. 8B schematically illustrates a cross-sectional view of a portion of another microneedle array 168 having a different embodiment of a calibration position 170. Each replaceable microneedle array 168 may include one or more calibration positions 170. Instead of a coated microneedle at the calibration position 170, this embodiment has a calibration protrusion 172 having a flat tipped portion 174 that can be actuated into contact with the reference analyte 166 without piercing the protective film 34. It can be appreciated that other geometries for the calibration protrusion are possible. It could also be useful that a replaceable coated microneedle array can include more than one calibration position, for example, in cases where biomedical measurements are made only infrequently.

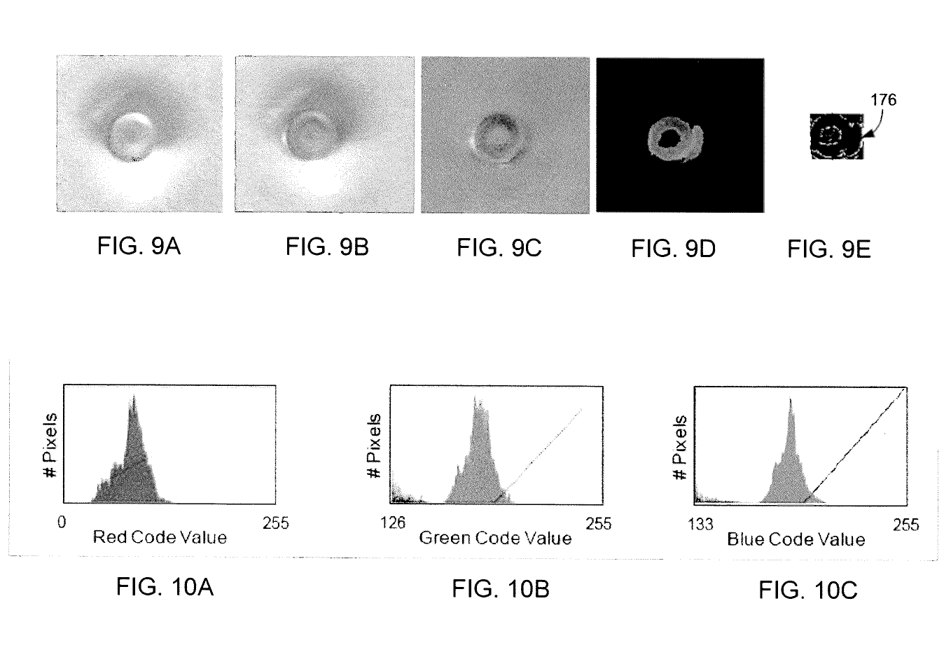

FIGS. 9A-9B show images captured by an image sensor showing a coated microneedle before and after insertion into a test environment, respectively. The image sensor may be operated in still or video acquisition modes, and the image sensor may include a CCD or CMOS imaging array sensor having multispectral capability, e.g. red (R), green (G) and blue (B) color channels. FIG. 9A shows an image of a microneedle having an outer coating that gives a color change in response to glucose, as viewed directly along the microneedle axis from its top, prior to insertion into a phantom skin model, the skin model having a glucose concentration beneath an upper membrane. When capturing this image, the microneedle was illuminated obliquely at about 45 degrees with white light, also from the top, although other embodiments may illuminate with other types of light, from other angles, and/or from more than one position. As just one example, the illumination could as well be along the direction of the microneedle axis from above the microneedle. Illumination from two or more oblique opposing directions as disclosed above is also desirable in some embodiments.

FIG. 9B shows the same microneedle after insertion into the phantom skin model having the glucose concentration and illustrates the associated color change. The change is seen only in a portion of the image field corresponding to the region of chemical sensing material which contacted the analyte. A computing device configured to execute a spatial image processing techniques was then employed to extract only the relevant portions of the image field to become the sampled area for the measurement. In this way, regions within the field which relate to irrelevant or erroneous portions may be eliminated from the data set. Erroneous portions may arise, for example, from defects in the analyte sensing material coated on the microneedle, or from interfering microstructures within the skin at the probe site, and the like. It is also possible by imaging the penetrated microneedle, to measure the diameter of the intersection of the skin and microneedle at the skin surface and thus to precisely determine the actual microneedle penetration depth. The depth determination can be used as well, to control the insertion depth, by adjusting the penetration depth. A dark ring can be noticed in the image both before and after insertion to the subject. The ring is not a fundamental problem but originates from limitations in illumination caused by total internal reflection when the sensing material is not in optical contact with the microneedle. Improved illumination profiles can virtually eliminate this artifact. Good adhesion of the sensing material along its entire interface with the needle eliminates the dark ring. The dark ring defect can also be excluded from the sampled data pixels.

FIG. 9C shows the background subtracted image, i.e. the difference image for before and after insertion. FIG. 9D defines only that portion of the image field having undergone a color change, and can be extracted to define the sample area for the measurement 176, shown as FIG. 9E.

FIGS. 10A-10C separately illustrate pixel histograms of the sampled color change area 176 for red, green, and blue channels, respectively. FIGS. 10A-10C also give the median and full-width at half-max (FWHM) values for the distributions shown in the histograms.

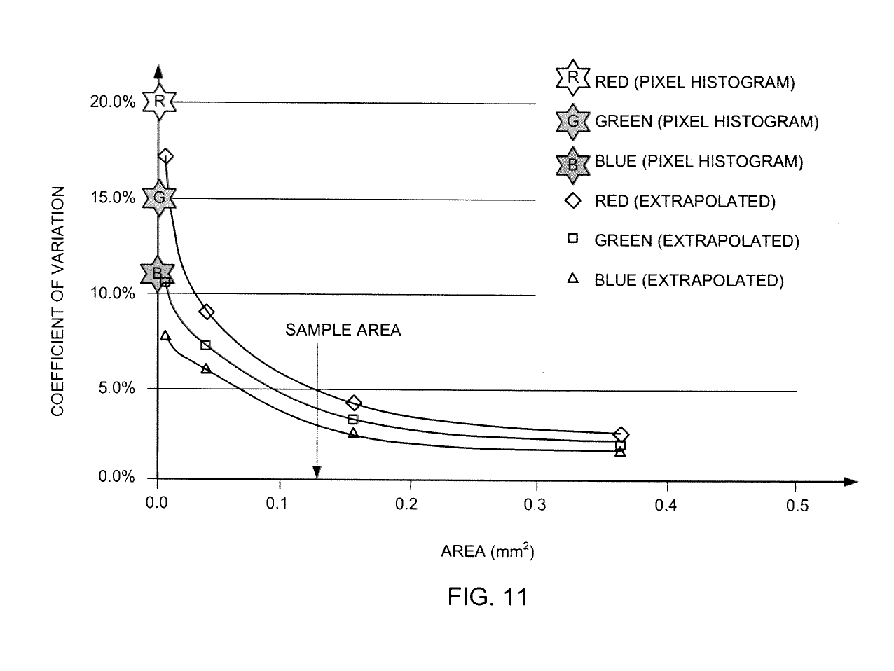

The distributions shown in FIGS. 10A-10B were used to derive the effect of sampled area size on the coefficient of variation (CV) in the intensity measurements in each color channel, as is given in FIG. 11. As shown, portions of the curves are from a fit to the data from the distributions and portions are extrapolated, based on the assumption that expected error will diminish according to the square root of the number of pixels employed in the measurement. In general, as expected, CV diminishes with increasing sampled area. It is a feature of image sensor arrays such as CCD and CMOS sensor arrays that very large numbers of pixels contribute to the data set used in the measurement of the bio-medically-relevant analyte.

When measuring intensity changes before and after exposure of the analyte to the chemical sensing material, it is important to account for the background signal of the initial condition. As an alternative to subtracting the background signal, a ratio of the initial intensity to the intensity after exposure to the analyte may be used. The logarithm of this ratio yields a quantity that is directly proportional to the analyte concentration. For example, in a glucose concentration measurement, if Cg is the glucose concentration, ∈g(Cg,λ) is the molar extinction coefficient of the colorant as a function of Cg and the wavelength of light, and Iₒᵤₜ is the measured output intensity, then:

log₁₀{Iₒᵤₜ(Cg=0,λ)/Iₒᵤₜ(Cg,λ)}=∈g(Cg,λ)(α)(Cg)(t_{eff})

where α is the yield of colorant per molecule of glucose, and t_{eff} is the effective optical thickness of the sensing material coating.

Thus, the log of the ratio of measured intensities before and after glucose exposure is a quantity directly proportional to glucose concentration. The log of the ratio of measured intensities is also generally a preferred computational method for analytes other than glucose, when using a change in the absorption of a colorant based on exposure to an analyte.

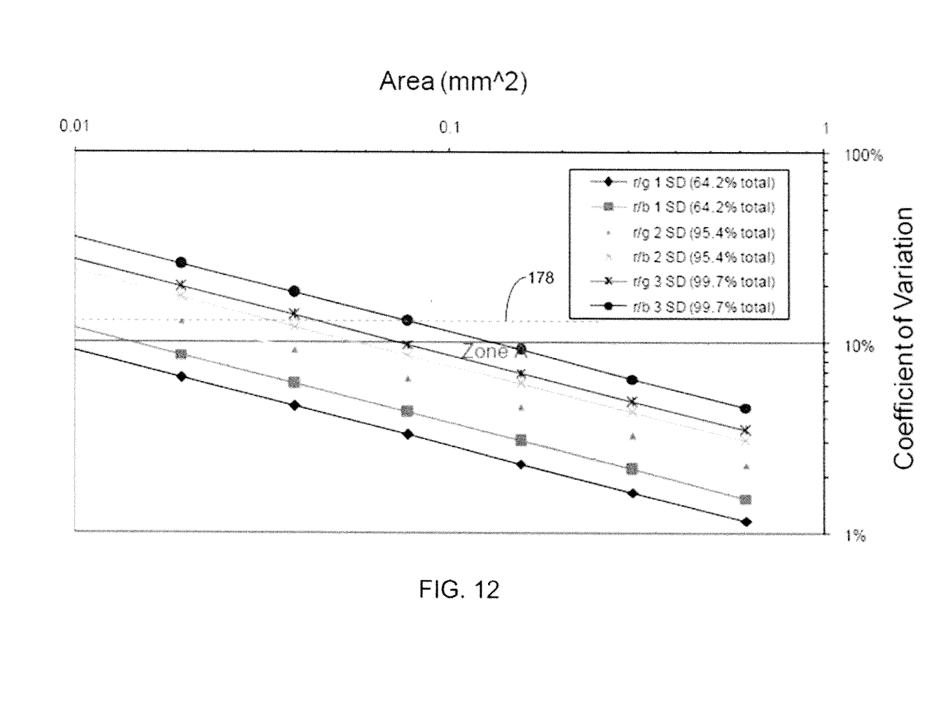

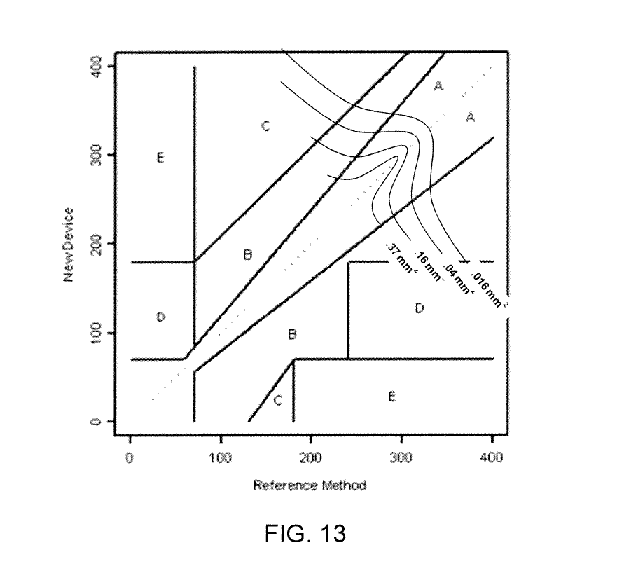

Though it is possible to determine analyte concentration by measuring changes in intensity within the sampled area before and after insertion of the coated microneedle, preferably as described above, it is further preferred that the change in color ratios (ratio of R/B, R/G, and G/B) be measured as well. By using ratios of response in different wavelengths, results are intrinsically normalized. FIG. 12 shows plots of color ratio CV computed from the data from the distributions of FIGS. 10A-10C that give the color ratio CV as a function of sampled area for various color ratio combinations. The color ratio CV vs. sampled area are given using sets of data points in the distributions of sampled data corresponding to 1, 2, and 3 standard deviations. Shown as well, is a line 178 corresponding to the boundary of the “A-zone” of the Clark Error Grid of FIG. 13. As can be seen, for the glucose concentration of the given measurement, and for a sampled area greater than about 0.04 mm², 99.7% of the pixel data points corresponding to the ratios R/B and R/G fall within the A-zone. FIG. 13 depicts a Clark Error Grid and shows the spread in data points derived from color ratio determinations for various sampled area sizes from FIG. 12.

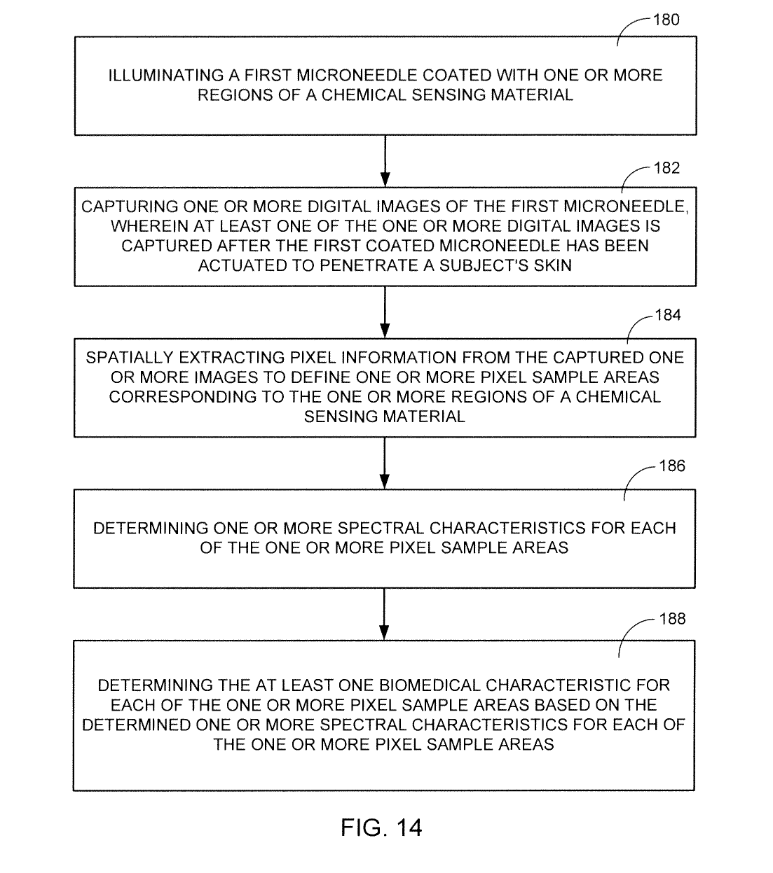

FIG. 14 illustrates one embodiment of a method for monitoring at least one biomedical characteristic. In step 180, a first microneedle coated with one or more regions of a chemical sensing material is illuminated. The illumination should occur at least while one or more digital images are captured in subsequent steps. One or more wavelengths may be chose for the illumination to highlight one or more aspects of the visible spectrum, the near infrared spectrum, ultraviolet spectrum, or other spectral regions. The one or more wavelengths of illumination may be chosen because they are of interest for the image capture and/or because they are of interest for causing fluorescence.

In step 182, one or more digital images of the first microneedle are captured, wherein at least one of the one or more digital images is captured after the first coated microneedle has been actuated to penetrate a subject's skin. The digital image capture may occur while the microneedle is still penetrating the subject's skin and/or after the microneedle has been extracted from the subject's skin. Optionally, at least another of the one or more digital images is captured before the first coated microneedle has been actuated to penetrate the subject's skin. Such a pre-penetration image can be used as a baseline image for later comparison.

In step 184, pixel information is spatially extracted from the captured one or more images to define one or more pixel sample areas corresponding to the one or more regions of a chemical sensing material. The one or more regions of chemical sensing material coated on the microneedle may be in known patterns/locations. Not every portion of the captured digital images needs to be evaluated or used. For example, in some embodiments, only pixels corresponding to known locations of the regions of chemical sensing material will be extracted and defined as the one or more pixel sample areas to be used for further analysis. In some embodiments, a pre-penetration image can be subtracted from a post-penetration image to subtract a background from consideration and to help more accurately define the one or more pixel sample areas. Preferably, the image used for background subtraction is captured at the moment that the coated microneedle is filled with fluid post penetration, but before any reaction with the analyte takes place.

In step 186, one or more spectral characteristics are determined for each of the one or more pixel sample areas. Each of the one or more pixel sample areas may correspond to a different region of chemical sensing material. Each region of chemical sensing material may be configured to react to different analytes or the same analyte, depending on the embodiment. In some embodiments, determining the one or more spectral characteristics for each of the one or more pixel sample areas can occur by determining a red pixel histogram, a green pixel histogram, and a blue pixel histogram for each of the one or more pixel sample areas. Such histograms may be compiled, for example, for reflected light exposures and fluorescence exposures and the histograms may be determined for each of the one or more captured digital images. The determined one or more spectral characteristics will be the basis for determining the at least one biomedical characteristic in a later step. In embodiments using histograms, the spectral characteristic determined from the histogram may include, but is not limited to an average, a window-average, a maximum, or a minimum. For example, in the case of glucose measurements, by being able to consider maxima for the spatially determined pixel sample area, the measurement can effectively filter out glucose measurements in areas where perhaps local cells have already started to consume the localized glucose, thereby avoiding data points which would tend to contribute to a less accurate glucose concentration measurement. In some embodiments, the determined one or more spectral characteristic is a ratio of measured intensities in different images. For example, an initial intensity may be determined from the pixel sample area of a digital image captured prior to insertion/penetration of the microneedle, or preferably, immediately after penetration. Then, a post-actuation intensity may be determined from the pixel sample area of a digital image captured after penetration of the microneedle and after such time as analyte-induced spectral changes have occurred. In some embodiments, the determined spectral characteristic may be the ratio of these two intensities.

In step 188, the at least one biomedical characteristic is determined for each of the one or more pixel sample areas based on the determined one or more spectral characteristics for each of the one or more pixel sample areas. In some embodiments, the at least one biomedical characteristic may be a concentration of an analyte. In such embodiments, the concentration may be determined by taking the log of the ratio of measured intensities described above. The log ratio of measured intensities may be proportional to a concentration of the target analyte in a predictable fashion as described previously. In some embodiments, rather than determining the at least one biomedical characteristic to be a concentration of an analyte, the at least one biomedical characteristic could be a true/false indicator for the presence of an analyte or a true/false indicator for the crossing of a threshold analyte level. Such non-limiting examples of biomedical characteristics may be determined, for example, in relation to glucose, cholesterol, HDL cholesterol, LDL cholesterol, alcohol, estrogen-progesterone, cortisol, a physiological chemical, and an exposed chemical.

Optionally, as discussed previously, an insertion depth may be determined for the microneedle based on the determined one or more spectral characteristics, or on the change in reflectivity induced by filling the porous layer with fluid, for each of the one or more pixel sample areas. Optionally, the at least one biomedical characteristic for each of the one or more pixel sample areas may be determined as a function of microneedle insertion depth. Furthermore, biomedical characteristics corresponding to insertion depths which are not of interest may be ignored to improve measurement accuracy.

Optionally, a calibration microneedle coated with one or more calibration regions of the chemical sensing material may be illuminated. One or more digital calibration images of the calibration microneedle may be captured, wherein at least one of the one or more digital calibration images is captured after the calibration microneedle has been actuated to contact a reference analyte. The digital calibration microneedle may be blunt in some embodiments. Pixel information may be spatially extracted from the captured one or more calibration images to define one or more calibration pixel sample areas corresponding to the one or more calibration regions of the chemical sensing material. One or more spectral calibration characteristics may be determined for each of the one or more calibration pixel sample areas. The determination of the at least one biomedical characteristic may be corrected for each of the one or more pixel sample areas based on the determined one or more spectral characteristics for each of the one or more pixel sample areas and the determined one or more spectral calibration characteristics.

Optionally, in some embodiments, an electronic medical record may be updated to include the determined at least one biomedical characteristic.

The methods disclosed herein, and their embodiments, may optionally be configured to check one or more microneedles of a microneedle array for evidence of prior use. For example, optionally, one or more screening digital images may be captured of the first microneedle (as well as any other or all microneedles of the microneedle array) prior to penetration of the subject's skin with the first microneedle. A used microneedle or microneedle array may have a pre-existing color change which can be detected and analyzed using the methods discloses above. For example, it may be determined whether or not the first microneedle has been previously used from a comparison of one or more spectral characteristics for each of one or more spatially extracted pixel sample areas, corresponding to the one or more regions of the chemical sensing material in the captured at least one screening digital image, with an expected standard. If it is determined that the first microneedle has been previously used, then the first microneedle may be prevented from penetrating the subject's skin. Additionally, the subject may be alerted if the at least one microneedle has previously been used.

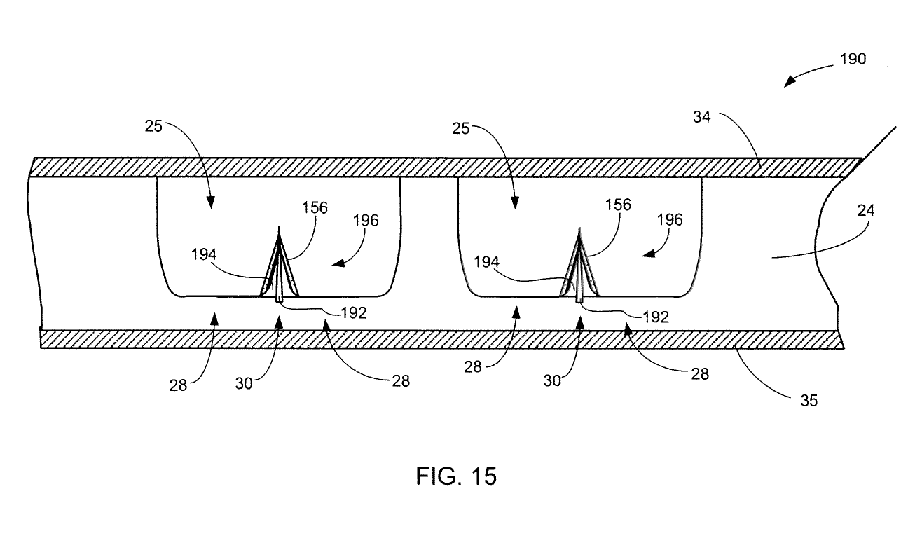

FIG. 15 schematically illustrates a cross-sectional view of a portion of another embodiment of a microneedle array 190. In accord with above descriptions of microneedle arrays, the microneedle array 190 may include a substrate 24 which has been micro-machined or precision molded to define one or more wells 25 and a corresponding microneedle base 30 supported by at least one restoring spring element 28. As described above, the substrate 24, at least in the area of the microneedle base 30 is preferably transparent or translucent. In this embodiment, a thin metal needle 192, for example an acupuncture needle, is embedded in the microneedle base 30 with the needle 192 protruding from the substrate. Each needle 192 is overmolded with a transparent or translucent polymer 194 to form a composite microneedle 196 having a metal core and a light transmissive tapered surrounding structure.

The one or more microneedles 196 can be coated with one or more regions of a chemical sensing material 156 that either changes its color or fluoresces or changes its fluorescence characteristics when in contact with one or more specific chemical species as discussed above. The chemical sensing material 156 may be optically transparent, reflective, opaque, or scattering.

In some embodiments, the microneedle array 190 may be sealed on at least a test-subject-facing side by a protective film 34. Other embodiments can also include a protective film 35 on the opposite side of the array 190 in order to seal the one or more microneedles 196 from interaction with the external environment and/or test subject, and in general to help maintain a sterile, dry environment for the one or more coated microneedles 196, prior to use. Some embodiments may also include a desiccant layer (not shown) on one or more of the protective films 34, 35 to provide a dry environment for the one or more coated microneedles 196. If a protective film 35 is used on the base side of the microneedle array 154, then the protective film is preferably transparent or translucent. Non-limiting examples for a protective films 34, 35 include polyvinylidene fluoride, polyvinyl chloride, polyvinylidene chloride, polypropylene, polyethylene terepthalate, polyethylene napthenate, ethylene-vinyl acetate copolymer, and low density polyethylene. The microneedle array 190 may be a removable and replaceable subassembly which the biomedical monitor 20 is configured to receive.

The embodiments of biomedical monitors disclosed herein, and their equivalents have a variety of advantages which have been discussed throughout the specification. The biomedical monitors may be removably attached to a subject and are able to make multiple sequential blood chemistry measurements. The biomedical monitor provides a highly useful device configuration and convenient fabrication process for dense arrays of individually actuated microneedles having integral chemical sensors. The compact wearable device can sample body chemistry without extracting a significant amount of blood or interstitial fluid either during or after the microneedle is inserted in the subject. Consequently, the degree of invasiveness and risk of contamination is reduced, while improving the hygiene of the process. Due to their high multiplicity, microneedles with integral chemical sensing material may be inserted in the subject in sequence over an extended period of time, each chemical sensing element being required to make measurements for only a short time period. The use of each microneedle for a limited time will eliminate the effect of bio-fouling. Sequential actuation of a multiple microneedles provides the ability for long term monitoring. Control of the serial actuation process can be programmed for a specific monitoring schedule, making the process practically continuous, if desired, and convenient for a subject. Due to their dense spacing and integrated actuation capability, many measurements may be made for extended time periods using a compact device worn by the subject as a small patch or chip. The biomedical monitor may be configured to sense chemicals which are naturally produced and/or found in a subject's body as well as chemicals which a subject has been exposed to, for example harmful toxins or biological components. The biomedical monitor may also be configured to receive a convenient replaceable microneedle array.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefor, is not intended to limit the claimed processes to any order except as may be specified in the claims. Accordingly, the invention is limited only by the following claims and equivalents thereto.

What is claimed is:

1. A replaceable microneedle array, comprising:
a plurality of moveable microneedles coated with at least one chemical sensing material coupled with a porous material;
a substrate defining wells to house the microneedles; and
at least one restoring spring element coupled between each microneedle and the substrate such that each of the plurality of microneedles is held at least partially in an associated well.

2. The replaceable microneedle array of claim 1, further comprising a protective film on a microneedle facing side of the array and covering the wells.

3. The replaceable microneedle array of claim 2, further comprising:
a calibration position comprising a moveable calibration protrusion coated with the at least one chemical sensing material; and
a patterned deposit of a non-volatile reference analyte on the protective film at a location corresponding to the calibration position.

4. The replaceable microneedle array of claim 3, wherein the calibration protrusion comprises a calibration microneedle.

5. The replaceable microneedle array of claim 1, wherein the substrate further defines the at least one restoring spring element coupled between each microneedle and the substrate.

6. The replaceable microneedle array of claim 1, wherein the substrate further comprises a material selected from the group consisting of silicon, silicon dioxide, silicon nitride, plastic, metal, glass, quartz, sapphire, and a dielectric material.

7. The replaceable microneedle array of claim 1, wherein at least one of the plurality of moveable microneedles coated with at least one chemical sensing material comprises a plurality of regions of chemical sensing material.

8. The replaceable microneedle array of claim 7, wherein:
at least two of the plurality of regions comprise different chemical sensing materials from each other.

9. The replaceable microneedle array of claim 1, wherein the at least one chemical sensing material comprises a medium which changes color when in contact with a target chemical specie.

10. The replaceable microneedle array of claim 1, wherein the at least one chemical sensing material comprises a medium which fluoresces when in contact with a target chemical specie.

11. The replaceable microneedle array of claim 1, wherein the at least one chemical sensing material comprises a medium which changes its fluorescence characteristics when in contact with a target chemical specie.

12. The replaceable microneedle array of claim 1, wherein the at least one chemical sensing medium comprises a material selected from the group consisting of glucose oxidase, peroxidase, glucose dehydrogenase, hexokinase-glucokinase, rhenium bipyridine, boronic acid having fluorophores, NBD-fluorophores, europium teracycline, oxidizable color-change dyes such as 4-aminoantipyrine, chromotropic acid, and potassium iodide in the presence of a tri-iodide ion host such as amylose, starch, polyvinyl acetate, polyvinyl alcohol, polyvinyl pyrrolidone, nylon, cellulose, and chitosan.

13. The replaceable microneedle array of claim 1, wherein at least one of the plurality of moveable microneedles comprises a capillary film.

14. The replaceable microneedle array of claim 1, wherein at least one of the plurality of moveable microneedles comprises at least one roughened surface.

15. The replaceable microneedle array of claim 1, wherein at least one of the plurality of moveable microneedles comprises a semi-permeable membrane overlay.

16. The replaceable microneedle array of claim 1, wherein at least one of the plurality of moveable microneedles comprises micro-particulate diffuse particles.

17. The replaceable microneedle array of claim 1, further comprising a backside film opposite a microneedle facing side of the array and covering the wells; and wherein:
the plurality of moveable microneedles are each coupled to the backside film; and
the backside film comprises the at least one restoring spring element coupled between each microneedle and the substrate.

18. The replaceable microneedle array of claim 1, wherein each of the microneedles comprises:
a metal needle embedded in a microneedle base defined by the substrate; and
a light-transmissive tapered structure surrounding at least a portion of the metal needle.
